SEQUENCE LISTING (31 sequences)

SEQ ID NO: 1 - DNA, 1104 bp, Mus musculus
atgaattccacagagtcctactttggaacggatgattatgacaacacagagtattattctattcctccagaccatgggccatgctccctagaagaggtcagaaacttcaccaaggtatttgtgccaattgcctactccttaatatgtgtctttggcctcctgggcaacattatggtggtgatgacctttgccttctacaagaaagccagatccatgactgacgtctacctgttgaacatggccatcacagacatactctttgtcctcaccctaccgttctgggcagttactcatgccaccaacacttgggttttcagcgatgcactgtgtaaactgatgaaaggcacatatgcggtcaactttaactgtgggatgctgctcctggcctgtatcagcatggaccggtacattgccatcgtccaggcaaccaaatctttccgggtacgctccagaacactgacgcacagtaaggtcatctgtgtggcagtgtggttcatctccatcatcatctcaagccctacatttatcttcaacaagaaatacgagctgcaggatcgtgatgtctgtgagccacggtacaggtctgtctcagagcccatcacgtggaagctgctgggtatgggactggagctgttctttgggttcttcacccctttgctgtttatggtgttctgctatctgttcattatcaagaccttggtgcaggcccagaactccaagaggcacagagccatccgagtcgtgatcgctgtggttctcgtgttcctggcttgtcagatccctcacaacatggtcctcctcgtgactgcggtcaacacgggcaaagtgggccggagctgcagcaccgagaaagtcctcgcctacaccaggaacgtggccgaggtcctggctttcctgcattgctgcctcaaccccgtgttgtatgcgtttattggacagaaattcagaaactacttcatgaagatcatgaaggatgtgtggtgtatgagaaggaagaataagatgcctggcttcctctgtgcccgggtttactcggaaagctacatctccaggcagaccagtgagaccgtcgaaaatgataatgcatcgtcctttaccatgtaa

SEQ ID NO: 2 - protein, 367 aa, Mus musculus
MNSTESYFGTDDYDNTEYYSIPPDHGPCSLEEVRNFTKVFVPIAYSLICVFGLLGNIMVVMTFAFYKKARSMTDVYLLNMAITDILFVLTLPFWAVTHATNTWVFSDALCKLMKGTYAVNFNCGMLLLACISMDRYIAIVQATKSFRVRSRTLTHSKVICVAVWFISIIISSPTFIFNKKYELQDRDVCEPRYRSVSEPITWKLLGMGLELFFGFFTPLLFMVFCYLFIIKTLVQAQNSKRHRAIRVVIAVVLVFLACQIPHNMVLLVTAVNTGKVGRSCSTEKVLAYTRNVAEVLAFLHCCLNPVLYAFIGQKFRNYFMKIMKDVWCMRRKNKMPGFLCARVYSESYISRQTSETVENDNASSFTM

SEQ ID NO: 3 - DNA, 1125 bp, Homo sapiens
atgagcggggaatcaatgaatttcagcgatgttttcgactccagtgaagattattttgtgtcagtcaatacttcatattactcagttgattctgagatgttactgtgctccttgcaggaggtcaggcagttctccaggctatttgtaccgattgcctactccttgatctgtgtctttggcctcctggggaatattctggtggtgatcacctttgctttttataagaaggccaggtctatgacagacgtctatctcttgaacatggccattgcagacatcctctttgttcttactctcccattctgggcagtgagtcatgccaccggtgcgtgggttttcagcaatgccacgtgcaagttgctaaaaggcatctatgccatcaactttaactgcgggatgctgctcctgacttgcattagcatggaccggtacatcgccattgtacaggcgactaagtcattccggctccgatccagaacactaccgcgcagcaaaatcatctgccttgttgtgtgggggctgtcagtcatcatctccagctcaacttttgtcttcaaccaaaaatacaacacccaaggcagcgatgtctgtgaacccaagtaccagactgtctcggagcccatcaggtggaagctgctgatgttggggcttgagctactctttggtttctttatccctttgatgttcatgatattttgttacacgttcattgtcaaaaccttggtgcaagctcagaattctaaaaggcacaaagccatccgtgtaatcatagctgtggtgcttgtgtttctggcttgtcagattcctcataacatggtcctgcttgtgacggctgcaaatttgggtaaaatgaaccgatcctgccagagcgaaaagctaattggctatacgaaaactgtcacagaagtcctggctttcctgcactgctgcctgaaccctgtgctctacgcttttattgggcagaagttcagaaactactttctgaagatcttgaaggacctgtggtgtgtgagaaggaagtacaagtcctcaggcttctcctgtgccgggaggtactcagaaaacatttctcggcagaccagtgagaccgcagataacgacaatgcgtcgtccttcactatgtga

SEQ ID NO: 4 - protein, 374 aa, Homo sapiens
MSGESMNFSDVFDSSEDYFVSVNTSYYSVDSEMLLCSLQEVRQFSRLFVPIAYSLICVFGLLGNILVVITFAFYKKARSMTDVYLLNMAIADILFVLTLPFWAVSHATGAWVFSNATCKLLKGIYAINFNCGMLLLTCISMDRYIAIVQATKSFRLRSRTLPRSKIICLVVWGLSVIISSSTFVFNQKYNTQGSDVCEPKYQTVSEPIRWKLLMLGLELLFGFFIPLMFMIFCYTFIVKTLVQAQNSKRHKAIRVIIAVVLVFLACQIPHNMVLLVTAANLGKMNRSCQSEKLIGYTKTVTEVLAFLHCCLNPVLYAFIGQKFRNYFLKILKDLWCVRRKYKSSGFSCAGRYSENISRQTSETADNDNASSFTM

SEQ ID NO: 5 - DNA, 20 bp, Artificial Sequence
  Oligonucleotide primer
ttgaaggacctgtggtgtgt

SEQ ID NO: 6 - DNA, 20 bp, Artificial Sequence
  oligonucleotide primer
ttgtcgttatctgcggtctc

SEQ ID NO: 7 - DNA, 20 bp, Artificial Sequence
  oligonucleotide probe
cctcccggcacaggagaagc

SEQ ID NO: 8 - DNA, 20 bp, Artificial Sequence
  oligonucleotide primer
ggtgagctggagtcatcaga

SEQ ID NO: 9 - DNA, 20 bp, Artificial Sequence
  oligonucleotide primer
gtgactctcaggcagtgctc

SEQ ID NO: 10 - DNA, 20 bp, Artificial Sequence
  oligonucleotide probe
ccttcagcctcactccgggc

SEQ ID NO: 11 - DNA, 20 bp, Artificial Sequence
  oligonucleotide primer
gaccagtgagaccgcagata

SEQ ID NO: 12 - DNA, 20 bp, Artificial Sequence
  oligonucleotide primer
tcacacatgccttagggaga

SEQ ID NO: 13 - DNA, 25 bp, Artificial Sequence
  oligonucleotide probe
cgacaatgcgtcgtccttcactatg

SEQ ID NO: 14 - DNA, 20 bp, Artificial Sequence
  oligonucleotide primer
gaggtcaggcagttctccag

SEQ ID NO: 15 - DNA, 20 bp, Artificial Sequence
  oligonucleotide primer
gctgccttgggtgttgtatt

SEQ ID NO: 16 - DNA, 20 bp, Artificial Sequence
  oligonucleotide primer
caggaggtcaggcagttctc

SEQ ID NO: 17 - DNA, 19 bp, Artificial Sequence
  oligonucleotide primer
ggctgcaatttgggtaaaa

SEQ ID NO: 18 - DNA, 20 bp, Artificial Sequence
  oligonucleotide primer
cacaggagaagcctgaggac

SEQ ID NO: 19 - DNA, 20 bp, Artificial Sequence
  oligonucleotide primer
gaggtcaggcagttctccag

SEQ ID NO: 20 - DNA, 20 bp, Artificial Sequence
  oligonucleotide primer
ggatggctttgtgcctttta

SEQ ID NO: 21 - DNA, 1852 bp, Homo sapiens
accgccgagaccgcgtccgccccgcgagcacagagcctcgcctttgccgatccgccgcccgtccacacccgccgccagctcaccatggatgatgatatcgccgcgctcgtcgtcgacaacggctccggcatgtgcaaggccggcttcgcgggcgacgatgccccccgggccgtcttcccctccatcgtggggcgccccaggcaccagggcgtgatggtgggcatgggtcagaaggattcctatgtgggcgacgaggcccagagcaagagaggcatcctcaccctgaagtaccccatcgagcacggcatcgtcaccaactgggacgacatggagaaaatctggcaccacaccttctacaatgagctgcgtgtggctcccgaggagcaccccgtgctgctgaccgaggcccccctgaaccccaaggccaaccgcgagaagatgacccagatcatgtttgagaccttcaacaccccagccatgtacgttgctatccaggctgtgctatccctgtacgcctctggccgtaccactggcatcgtgatggactccggtgacggggtcacccacactgtgcccatctacgaggggtatgccctcccccatgccatcctgcgtctggacctggctggccgggacctgactgactacctcatgaagatcctcaccgagcgcggctacagcttcaccaccacggccgagcgggaaatcgtgcgtgacattaaggagaagctgtgctacgtcgccctggacttcgagcaagagatggccacggctgcttccagctcctccctggagaagagctacgagctgcctgacggccaggtcatcaccattggcaatgagcggttccgctgccctgaggcactcttccagccttccttcctgggcatggagtcctgtggcatccacgaaactaccttcaactccatcatgaagtgtgacgtggacatccgcaaagacctgtacgccaacacagtgctgtctggcggcaccaccatgtaccctggcattgccgacaggatgcagaaggagatcactgccctggcacccagcacaatgaagatcaagatcattgctcctcctgagcgcaagtactccgtgtggatcggcggctccatcctggcctcgctgtccaccttccagcagatgtggatcagcaagcaggagtatgacgagtccggcccctccatcgtccaccgcaaatgcttctaggcggactatgacttagttgcgttacaccctttcttgacaaaacctaacttgcgcagaaaacaagatgagattggcatggctttatttgttttttttgttttgttttggttttttttttttttttggcttgactcaggatttaaaaactggaacggtgaaggtgacagcagtcggttggagcgagcatcccccaaagttcacaatgtggccgaggactttgattgcacattgttgtttttttaatagtcattccaaatatgagatgcgttgttacaggaagtcccttgccatcctaaaagccaccccacttctctctaaggagaatggcccagtcctctcccaagtccacacaggggaggtgatagcattgctttcgtgtaaattatgtaatgcaaaatttttttaatcttcgccttaatacttttttattttgttttattttgaatgatgagccttcgtgcccccccttcccccttttttgtcccccaacttgagatgtatgaaggcttttggtctccctgggagtgggtggaggcagccagggcttacctgtacactgacttgagaccagttgaataaaagtgcacaccttaaaaatgaaaaaaaaaaaaaaaaaaaaaaaaaaaaaaaaaaaaaaaaa

SEQ ID NO: 22 - DNA, 20 bp, Artificial Sequence
  oligonucleotide primer
tggacttcgagcaagagatg

SEQ ID NO: 23 - DNA, 20 bp, Artificial Sequence
  oligonucleotide primer
gaaggaaggctggaagagtg

SEQ ID NO: 24 - DNA, 20 bp, Artificial Sequence
  oligonucleotide probe
cggctgcttccagctcctcc

SEQ ID NO: 25 - DNA, 18 bp, Artificial Sequence
  oligonucleotide primer
gcacccagcacaatgaag

SEQ ID NO: 26 - DNA, 20 bp, Artificial Sequence
  oligonucleotide primer
cgatccacacggagtacttg

SEQ ID NO: 27 - DNA, 24 bp, Artificial Sequence
  oligonucleotide probe
caagatcattgctcctcctgagcg

SEQ ID NO: 28 - DNA, 19 bp, Artificial Sequence
  oligonucleotide primer
ggcatgggtcagaaggatt

SEQ ID NO: 29 - DNA, 20 bp, Artificial Sequence
  oligonucleotide primer
agaaggtgtggtgccagatt

SEQ ID NO: 30 - DNA, 20 bp, Artificial Sequence
  oligonucleotide probe
catcgagcacggcatcgtca

SEQ ID NO: 31 - DNA, 851 bp, Homo sapiens
agaatataacagcactcccaaagaactgggtactcaacactgagcagatctgttctttgagctaaaaaccatgtgctgtaccaagagtttgctcctggctgctttgatgtcagtgctgctactccacctctgcggcgaatcagaagcagcaagcaactttgactgctgtcttggatacacagaccgtattcttcatcctaaatttattgtgggcttcacacggcagctggccaatgaaggctgtgacatcaatgctatcatctttcacacaaagaaaaagttgtctgtgtgcgcaaatccaaaacagacttgggtgaaatatattgtgcgtctcctcagtaaaaaagtcaagaacatgtaaaaactgtggcttttctggaatggaattggacatagcccaagaacagaaagaaccttgctggggttggaggtttcacttgcacatcatggagggtttagtgcttatctaatttgtgcctcactggacttgtccaattaatgaagttgattcatattgcatcatagtttgctttgtttaagcatcacattaaagttaaactgtattttatgttatttatagctgtaggttttctgtgtttagctatttaatactaattttccataagctattttggtttagtgcaaagtataaaattatatttgggggggaataagattatatggactttcttgcaagcaacaagctattttttaaaaaaaactatttaacattcttttgtttatattgttttgtctcctaaattgttgtaattgcattataaaataagaaaaatattaataagacaaatattgaaaataaagaaacaaaaagttcttctgttaaaaaaaaa

(19) United States
(12) Patent Application Publication
(10) Pub. No.: US 2013/0316338 A1
(43) Pub. Date: Nov. 28, 2013

(54) CCR6 As A Biomarker of Alzheimer's Disease

(71) Applicants: Halina Offner-Vandenbark, Portland, OR (US); Joseph F. Quinn, Portland, OR (US); Sandhya Subramanian, Portland, OR (US)

(72) Inventors: Halina Offner-Vandenbark, Portland, OR (US); Joseph F. Quinn, Portland, OR (US); Sandhya Subramanian, Portland, OR (US)

(73) Assignees: The United States Government as Represented by the Department of Veterans Affairs, Washington, DC (US); Oregon Health & Science University, Portland, OR (US)

(21) Appl. No.: 13/806,394

(22) PCT Filed: Jun. 28, 2011
(86) PCT No.: PCT/US11/42114

§ 371 Date: Mar. 28, 2013

Related U.S. Application Data
(60) Provisional application No. 61/359,760, filed on Jun. 29, 2010.

(51) Int. Cl. C12Q 1/68; G01N 33/68

(52) U.S. Cl. CPC C12Q 1/6883; G01N 33/6896
USPC 435/6.11; 435/6.12; 435/7.21

(57) ABSTRACT

Disclosed are methods used to diagnose Alzheimer's disease (AD) in a subject. The methods involve determining the amount of chemokine receptor 6 (CCR6) expressed in a biological sample. Expression of CCR6 in the sample that exceeds a threshold level of expression signifies that the subject has AD, even if the subject has not yet developed symptoms of AD. The methods may also be used to monitor the effectiveness of an AD treatment. Kits that facilitate the use of the methods are also disclosed.

### PRIORITY CLAIM

This application claims priority to U.S. Application No. 61/359,760, filed 29 Jun. 2010 which is hereby incorporated by reference in its entirety.

### ACKNOWLEDGMENT OF GOVERNMENT SUPPORT

This invention was made with United States government support pursuant to Grant Nos. NS45445 and NS47661 awarded by the National Institutes of Health (NIH) and the Department of Veterans Affairs Merit review and Biomedical Laboratory R & D Service. The United States Government has certain rights in the invention.

### FIELD

This disclosure relates to the field of Alzheimer's disease biomarkers and specifically, to methods of diagnosing Alzheimer's disease using the expression of chemokine receptor 6 (CCR6).

### BACKGROUND

Alzheimer's disease (AD) is the leading cause of dementia in elderly populations throughout the world with more than 35 million people worldwide having AD. AD is characterized by a deterioration of memory and other cognitive functions. AD is associated with the deposition in brain tissue of misfolded β-amyloid (Aβ) originated from proteolysis of the amyloid precursor protein (APP) by several enzymes, including presenilin-1. Pathologic forms of Aβ include soluble oligomers and insoluble Aβ plaques, which are surrounded by activated microglia, reactive astrocytes, and dystrophic neurites. In addition, numerous neurons in the cerebral cortex and subcortical nuclei accumulate neurofibrillary tangles made of paired helical filaments derived from the cytoskeletal protein tau (for a review see Querfurth and LaFerla, N Engl J Med 362, 329-344 (2010)).

Inflammation associated with glial activation and both synaptic and neuronal losses are also characteristics of AD. For example, chronically activated microglia release IL-1, IL-6, and TNF-α (Meda et al., J Neuroimmunol 93, 45-52 (1999); Janelsins et al. J Neuroinflammation 2, 23-28 (2005)) and express receptors for Aβ oligomers triggering the release of cytokines, glutamate, and nitric oxide (Yan et al., Nature 382, 685-691 (1996); Li et al., J Neurosci 23, 1605-1611 (2003)). Inflammatory cells such as monocytes have been shown to migrate from the peripheral blood into the brain of AD patients (Fiala et al. Mol Med. 4, 480-489 (1998)). The use of anti-inflammatory drugs such as (NSAIDs) have been shown to reduce the risk of developing AD. This suggests that inflammatory mechanisms may play a role in AD pathogenesis. That said, trials of NSAIDs in AD have failed to modify clinical outcomes in patients displaying AD symptoms. This suggests strongly that treatment of AD is more likely to be effective if the treatment begins prior to the subject displaying clinical signs. Currently, no test that identifies the presence of AD in presymptomatic subjects is available to clinicians. It is therefore beneficial to identify biomarkers that identify patients with AD prior to the development of symptoms.

The inflammatory response in the brains of mice with experimentally induced AD-like disease has been investigated (El Khoury J B et al, J Exp Med 197, 1657-1666 (2003) Lee J W et al, J Neuroinflammation 5, 37-50 (2008)) little is known about the role of chemokines and chemokine receptors in this process. Higher CCR6 RNA expression relative to normal has been seen in brain tissue (particularly the hippocampus) of mice with experimentally induced AD like disease (Jee S W et al, Neurochem Res 31, 1042-1052 (2006)). The Jee reference does not teach that CCR6 is differentially expressed in any tissue other than brain tissue. Neither does the Jee reference teach that CCR6 overexpression in mice with experimentally induced AD-like disease correlates with a diagnosis of AD in humans.

Currently, a definite diagnosis of AD requires tissue examination at autopsy or biopsy. However, a diagnosis can be made with high accuracy by using clinical criteria (see McKhann G et al, Neurology 34, 939-944 (1984) hereby incorporated by reference in its entirety.) There are no molecular tests available to clinicians that provide a diagnosis of Alzheimer's disease using an easily accessible bi0logical sample site. A minimally invasive molecular test that identifies a patient suffering from AD—especially one that identified such a patient prior to the onset of symptoms, would provide invaluable information to patients, physicians, researchers, and care providers and could inform treatment decisions for this disease.

### SUMMARY

There is an urgent need for tools for diagnosing AD or the predisposition to developing Alzheimer's disease prior to the onset of the clinical symptoms of AD, so that subjects can benefit from early intervention. It is disclosed herein that a method that determines the expression level of CCR6 in a subject can be used to diagnose AD in that subject.

One embodiment of the invention involves determining the expression level of CCR6 mRNA and/or CCR6 protein in a biological sample from the subject and comparing the expression level of CCR6 in the sample to a threshold level of CCR6 expression. CCR6 expression in the sample that exceeds the threshold level of CCR6 expression signifies that the subject has AD.

The expression level of CCR6 mRNA can be determined by any appropriate method of assessing mRNA expression, including reverse transcription polymerase chain reaction and TaqMan® reverse transcription polymerase chain reaction, among others.

The expression level of CCR6 protein can be determined by any appropriate method of assessing protein expression including methods that involve a reagent capable of specifically binding CCR6 protein, such as labeled or unlabeled antibodies. The expression level of CCR6 protein may also be determined through the use of methods that do not require the use of specific binding agents. Such methods include mass spectrometry and gel electrophoresis (among others).

The subject may be any appropriate subject, such as a human patient. The subject may have a genomic polymorphism that indicates a predisposition to developing AD such as the ApoE4 allele. Additionally, the subject may or may not display AD symptoms at the time that CCR6 expression is determined.

The threshold level of expression will be different for every method of determining CCR6 expression, but one of skill in the art will understand how to determine the threshold level of expression for any particular method of determining CCR6 expression, especially in light of this disclosure.

The biological sample may be derived from any site that comprises mononuclear cells. Preferably, the biological sample is a readily accessible biological sample, such as blood, rather than a less accessible biological sample, such as brain. In some aspects of the invention, the biological sample comprises whole blood. In those cases, any component or fraction of blood, such as mononuclear cells or CD19+ B cells may be used as the biological sample.

Another embodiment of the invention involves monitoring a subject's response to an Alzheimer's disease treatment. This involves obtaining at least two biological samples from the subject: a first sample obtained prior to the treatment, a second sample obtained following the treatment. CCR6 expression is determined in both samples. A decrease in CCR6 expression from the first sample to the second sample is an indication that the treatment is effective and an increase or maintenance of CCR6 expression from the first sample to the second sample is an indication that the treatment is ineffective. This method may be used to test the effectiveness of any AD treatment, including experimental pharmaceutical compositions or approved pharmaceutical compositions.

Another embodiment of the invention involves a kit that facilitates the diagnosis of Alzheimer's disease in a subject based upon a determination of the expression of CCR6 in a biological sample from the subject. Components of the kit include a reagent capable of specific binding to CCR6 mRNA or CCR6 protein and an indication of a threshold level of expression of CCR6. CCR6 expression in a sample from a subject that exceeds the threshold level of CCR6 expression signifies that the subject has AD. The reagent may comprise a nucleic acid capable of binding to all or part of CCR6 mRNA or cDNA. The reagent may comprise a protein that binds CCR6, such as an anti-CCR6 antibody. The reagent may also comprise a label. In the aspect of the invention in which the reagent comprises a label, the kit may further comprise a second reagent capable of binding the label. The indication of the threshold of expression may be anything that communicates the threshold level of expression to the end user of the kit. The indication may be a numerical value or a control that yields a result similar to that of a sample at the threshold level of expression.

The foregoing and other features of the disclosure will become more apparent from the following detailed description of several embodiments which proceeds with reference to the accompanying figures.

### BRIEF DESCRIPTION OF THE DRAWINGS

FIG. 1 is a set of bar graphs depicting the percentages of cells expressing the indicated biomarker proteins on cells collected from the spleens of 12-15 month old 3×-transgenic Alzheimer's disease (3×Tg-AD) mice. Wild-type results are from age-matched controls.

FIG. 2 is a set of bar graphs depicting the percentages of cells expressing the indicated biomarker proteins on cells collected from the spleens of 5-6 month old 3×Tg-AD mice. Wild type results are from age- and gender-matched controls.

FIG. 3 is a flow cytometry plot and a bar graph, both depicting the distribution of cells expressing the biomarker proteins Gr-1 and CD45 collected from the brains of 5-6 month old 3×Tg-AD mice. Wild-type results are from age-matched controls.

FIG. 4A is a bar graph depicting the expression of the indicated cytokine proteins in ex vivo cultures of splenocytes collected from 12-15 month old female 3×Tg-AD mice. Wild-type results are from age- and gender-matched controls.

FIG. 4B is a bar graph depicting the expression of the indicated cytokine proteins in ex vivo cultures of splenocytes collected from 5-6 month old female 3×Tg-AD mice. Wild-type results are from age- and gender-matched controls.

FIG. 4C is a bar graph depicting the expression of the indicated cytokine proteins in ex vivo cultures of splenocytes collected from 5-6 month old male 3×Tg-AD mice. Wild-type results are from age- and gender-matched controls.

FIG. 5A is a bar graph depicting the expression of the indicated biomarker RNA in brain mononuclear cells collected from 12-15 month old female 3×Tg-AD mice. Wild-type results are from age- and gender-matched controls.

FIG. 5B is a bar graph depicting the expression of the indicated biomarker RNA in brain mononuclear cells collected from 5-6 month old female 3×Tg-AD mice. Wild-type results are from age- and gender-matched controls.

FIG. 5C is a bar graph depicting the expression of the indicated biomarker RNA in brain mononuclear cells collected from 5-6 month old male 3×Tg-AD mice. Wild-type results are from age- and gender-matched controls.

FIG. 6A is a bar graph depicting the expression of the indicated biomarker RNA in whole spleens collected from 12-15 month old female 3×Tg-AD mice. Wild type results are from age- and gender-matched controls.

FIG. 6B is a bar graph depicting the expression of the indicated biomarker RNA in whole spleens collected from 5-6 month old female 3×Tg-AD mice. Wild type results are from age- and gender-matched controls.

FIG. 6C is a bar graph depicting the expression of the indicated biomarker RNA in whole spleens collected from 5-6 month old male 3×Tg-AD mice. Wild type results are from age- and gender-matched controls.

FIG. 7 is a bar graph depicting the expression of CCR6 mRNA in peripheral blood mononuclear cells collected from female human patients diagnosed as suffering from Alzheimer's disease (AD) compared to age- and gender-matched healthy controls (HC)

### SEQUENCE LISTING

The nucleic and amino acid sequences listed in the sequence listing are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. In the accompanying sequence listing:

SEQ ID NO: 1 is an exemplary nucleotide sequence encoding a murine CCR6 receptor.

SEQ ID NO: 2 is an amino acid sequence for an exemplary murine CCR6 receptor.

SEQ ID NO: 3 is an exemplary nucleotide sequence encoding a human CCR6 receptor.

SEQ ID NO: 4 is an amino acid sequence for an exemplary human CCR6 receptor.

SEQ ID NO: 5 is an oligonucleotide primer sequence that may be used in the amplification of CCR6.

SEQ ID NO: 6 is an oligonucleotide primer sequence that may be used in the amplification of CCR6.

SEQ ID NO: 7 is an oligonucleotide probe sequence that may be used in the detection of CCR6, for example in quantitative PCR.

SEQ ID NO: 8 is an oligonucleotide primer sequence that may be used in the amplification of CCR6.

SEQ ID NO: 9 is an oligonucleotide primer sequence that may be used in the amplification of CCR6.

SEQ ID NO: 10 is an oligonucleotide probe sequence that may be used in the detection of CCR6, for example in quantitative PCR.

SEQ ID NO: 11 is an oligonucleotide primer sequence that may be used in the amplification of CCR6.

SEQ ID NO: 12 is an oligonucleotide primer sequence that may be used in the amplification of CCR6.

SEQ ID NO: 13 is an oligonucleotide probe sequence that may be used in the detection of CCR6, for example in quantitative PCR.

SEQ ID NO: 14 is an oligonucleotide primer sequence that may be used in the amplification of CCR6.

SEQ ID NO: 15 is an oligonucleotide primer sequence that may be used in the amplification of CCR6.

SEQ ID NO: 16 is an oligonucleotide primer sequence that may be used in the amplification of CCR6.

SEQ ID NO: 17 is an oligonucleotide primer sequence that may be used in the amplification of CCR6.

SEQ ID NO: 18 is an oligonucleotide primer sequence that may be used in the amplification of CCR6.

SEQ ID NO: 19 is an oligonucleotide primer sequence that may be used in the amplification of CCR6.

SEQ ID NO: 20 is an oligonucleotide primer sequence that may be used in the amplification of CCR6.

SEQ ID NO: 21 is an exemplary nucleotide sequence encoding a human β-actin

SEQ ID NO: 22 is an oligonucleotide primer sequence that may be used in the amplification of β-actin.

SEQ ID NO: 23 is an oligonucleotide primer sequence that may be used in the amplification of β-actin.

SEQ ID NO: 24 is an oligonucleotide probe sequence that may be used in the detection of β-actin, for example in quantitative PCR.

SEQ ID NO: 25 is an oligonucleotide primer sequence that may be used in the amplification of β-actin.

SEQ ID NO: 26 is an oligonucleotide primer sequence that may be used in the amplification of β-actin.

SEQ ID NO: 27 is an oligonucleotide probe sequence that may be used in the amplification of β-actin.

SEQ ID NO: 28 is an oligonucleotide primer sequence that may be used in the amplification of β-actin.

SEQ ID NO: 29 is an oligonucleotide primer sequence that may be used in the amplification of β-actin.

SEQ ID NO: 30 is an oligonucleotide probe sequence that may be used in the amplification of β-actin.

SEQ ID NO: 31 is an amino acid sequence for an exemplary human CCL20 protein.

### DETAILED DESCRIPTION

### I. Terms

The following explanations of terms and methods are provided to better describe the present disclosure and to guide those of ordinary skill in the art in the practice of the present disclosure.

Administration:

To provide or give a subject an agent, such as a treatment for Alzheimer's disease agent, by any effective route. Exemplary routes of administration include, but are not limited to, injection (such as subcutaneous, intramuscular, intradermal, intraperitoneal, and intravenous), oral, sublingual, rectal, transdermal, intranasal, vaginal and inhalation routes.

Alzheimer's disease (AD):

A progressive brain disorder that occurs gradually and results in memory loss, behavioral and personality changes, and a decline in mental abilities. These losses are related to the death of brain cells and the breakdown of the connections between them. The course of this disease varies from person to person, as does the rate of decline. On average, AD patients live for 8 to 10 years after they are diagnosed, though the disease can last up to 20 years. AD advances by stages, from early, mild forgetfulness to a severe loss of mental function. At first, AD destroys neurons in parts of the brain that control memory, especially in the hippocampus and related structures. As nerve cells in the hippocampus stop functioning properly, short-term memory fails. AD also attacks the cerebral cortex, particularly the areas responsible for language and reasoning.

Dementias of all types including, but not limited to, AD result in progressive deterioration in the functioning of the subject and result in steadily worsening behavioral problems that coincide with the deterioration in cognitive functioning and are part of the same disease process. Typical behavioral problems shown by subjects with AD include, but are not limited to, depression, psychosis, delusions, sleep disturbance, wandering, anger outbursts, aggression, agitation, apathy, anxiety, suspiciousness, fearfulness and paranoia. In the final stages of most forms of AD, including AD, victims are bedridden, lose urinary and bowel control and suffer epileptic attacks. Death is usually due to pneumonia or urinary tract infection.

The clinical manifestations of AD are fairly characteristic, memory disturbance occurs early in the disease; subjects have difficulty learning and remembering new material. Spatial and temporal disorientation also may occur early, with subjects becoming lost in familiar surroundings. Aphasia, apraxia and acalculia develop as the disease progresses, and apathy or paranoia may occur. Subjects often have delusions of theft and spousal infidelity. Subjects may wander, pace, open and close drawers repeatedly, and repeat the same questions. Sleep-wake cycle abnormalities may become evident; for example, a subject may be awake at night but think that it is daytime. Activities of daily living decline throughout the illness. Subjects lose the ability to eat and groom themselves and have difficulty dressing. In the terminal stages of the disease, subjects exhibit cognitive decline in virtually all intellectual spheres, motor abnormalities become evident and both urinary and fecal incontinence develops.

A feature of AD is the development of multiple cognitive deficits that include memory impairment and at least one of the following cognitive disturbances: aphasia, apraxia, agnosia or a disturbance in executive functioning. The cognitive deficits must be sufficiently severe to cause impairment in occupational or social functioning and must represent a decline from a previously higher level of functioning.

Memory impairment is required to make the diagnosis of AD and is a prominent early symptom. Individuals with AD become impaired in their ability to learn new material, or they forget previously learned material. Most individuals with AD have both forms of memory impairment, although it is sometimes difficult to demonstrate the loss of previously learned material early in the course of the disorder. They may lose valuables like wallets and keys, forget food cooking on the stove, and become lost in unfamiliar neighborhoods. In advanced stages of AD, memory impairment is so severe that the person forgets his or her occupation, schooling, birthday, family members and sometimes even name.

Memory may be formally tested by asking the person to register, retain, recall and recognize information. The ability to learn new information may be assessed by asking the individual to learn a list of words. The individual is requested to repeat the words (registration), to recall the information after a delay of several minutes (retention, recall), and to recognize the words from a multiple list (recognition). Individuals with difficulty learning new information are not helped by clues or prompts, e.g., multiple-choice questions, because they did not learn the material initially. In contrast, individuals with primarily retrieval deficits can be helped by clues and prompts because their impairment is in the ability to access their memories. Remote memory may be tested by asking the individual to recall personal information or past material that the individual found of interest, e.g., politics, sports, entertainment. It is also useful to determine (from the individual and informants) the impact of the memory disturbances on the individual's functioning, e.g., ability to work, shop, cook, pay bills, return home without getting lost.

Deterioration of language function (aphasia) may be manifested by difficulty producing the names of individuals and objects. The speech of individuals with aphasia may become vague or empty, with long circumlocutory phrases and excessive use of terms of indefinite reference, such as “thing” and “it”. Comprehension of spoken and written language and repetition of language may also be compromised. In the advanced stages of AD, individuals may be mute or have a deteriorated speech pattern characterized by echolalia, i.e., echoing what is heard; or palilalia, i.e., repeating sounds or words over and over. Language is tested by asking the individual to name objects in the room, e.g., tie, dress, desk, lamp; or body parts, e.g., nose, chin, shoulder, follow commands, e.g., “point at the door and then at the table”; or repeat phrases, e.g., “no ifs, ands or buts”.

Amplifying a Nucleic Acid Molecule:

To increase the number of copies of a nucleic acid molecule, such as a gene or fragment of a gene, for example a region of a gene that encodes a Alzheimer's disease biomarker, such as chemokine receptor 6 (CCR6). The resulting products are called amplification products.

An example of in vitro amplification is the polymerase chain reaction (PCR). Other examples of in vitro amplification techniques include quantitative real-time PCR, strand displacement amplification (see U.S. Pat. No. 5,744,311); transcription-free isothermal amplification (see U.S. Pat. No. 6,033,881); repair chain reaction amplification (see WO 90/01069); ligase chain reaction amplification (see EP-A-320 308); gap filling ligase chain reaction amplification (see U.S. Pat. No. 5,427,930); coupled ligase detection and PCR (see U.S. Pat. No. 6,027,889); and NASBA™ RNA transcription-free amplification (see U.S. Pat. No. 6,025,134).

A commonly used method for real-time quantitative polymerase chain reaction involves the use of a double stranded DNA dye (such as SYBR Green I dye). For example, as the amount of PCR product increases, more SYBR Green I dye binds to DNA, resulting in a steady increase in fluorescence. Another commonly used method is real-time quantitative TaqMan® PCR (Applied Biosystems). This type of PCR has reduced the variability traditionally associated with quantitative PCR, thus allowing the routine and reliable quantification of PCR products to produce sensitive, accurate, and reproducible measurements of levels of gene expression. The 5′ nuclease assay provides a real-time method for detecting only specific amplification products. During amplification, annealing of the probe to its target sequence generates a substrate that is cleaved by the 5′ nuclease activity of Taq DNA polymerase when the enzyme extends from an upstream primer into the region of the probe. This dependence on polymerization ensures that cleavage of the probe occurs only if the target sequence is being amplified. The use of fluorogenic probes makes it possible to eliminate post-PCR processing for the analysis of probe degradation. The probe is an oligonucleotide with both a reporter fluorescent dye and a quencher dye attached. While the probe is intact, the proximity of the quencher greatly reduces the fluorescence emitted by the reporter dye by Förster resonance energy transfer (FRET) through space. Probe design and synthesis has been simplified by the finding that adequate quenching is observed for probes with the reporter at the 5′ end and the quencher at the 3′ end.

Antibody:

A polypeptide ligand comprising at least a light chain or heavy chain immunoglobulin variable region which specifically recognizes and binds an epitope of an antigen, such as an Alzheimer's disease biomarker, for example CCR6, or a fragment thereof. Antibodies can be composed of a heavy and a light chain, each of which has a variable region, termed the variable heavy (VH) region and the variable light (VL) region. Together, the VH region and the VL region are responsible for binding the antigen recognized by the antibody. This includes intact immunoglobulins and the variants and portions of them well known in the art, such as Fab′ fragments, F(ab)′2 fragments, single chain Fv proteins (“scFv”), and disulfide stabilized Fv proteins (“dsFv”). The term also includes recombinant forms such as chimeric antibodies (for example, humanized murine antibodies), heteroconjugate antibodies (such as, bispecific antibodies). See also, Pierce Catalog and Handbook, 1994-1995 (Pierce Chemical Co., Rockford, Ill.); Kuby, Immunology, 3rd Ed., W.H. Freeman & Co., New York, 1997. Exemplary antibodies that specifically bind to CCR6 protein are commercially available. In some examples an antibody is a monoclonal antibody. In some examples an antibody is a polyclonal antibody.

B Cell:

A lymphocyte, a type of white blood cell (leukocyte), that develops into a plasma cell (a “mature B cell”), which produces antibodies. An “immature B cell” is a cell that can develop into a mature B cell. Generally, pro-B cells (that express, for example, CD45 or B220) undergo immunoglobulin heavy chain rearrangement to become pro B or pre B cells, and further undergo immunoglobulin light chain rearrangement to become an immature B cells. In some examples, B cells express the cell surface marker CD19, and can be termed CD19+ B cells.

Binding or Stable Binding:

An association between two substances or molecules, such as the association of an antibody with a peptide (such as a CCR6 peptide), nucleic acid to another nucleic acid (such as the binding of a probe to CCR6 RNA or CCR6 cDNA), or the association of a protein with another protein or nucleic acid molecule. Binding can be detected by any procedure known to one skilled in the art, for example in the case of a nucleic acid encoding a CCR6 protein, such as by physical or functional properties of the target:oligonucleotide complex.

Physical methods of detecting the binding of complementary strands of nucleic acid molecules, include but are not limited to, such methods as DNase I or chemical footprinting, gel shift and affinity cleavage assays, Northern blotting, dot blotting and light absorption detection procedures. For example, one method involves observing a change in light absorption of a solution containing an oligonucleotide (or an analog) and a target nucleic acid at 220 to 300 nm as the temperature is slowly increased. If the oligonucleotide or analog has bound to its target, there is a sudden increase in absorption at a characteristic temperature as the oligonucleotide (or analog) and target disassociate from each other, or melt. In another example, the method involves detecting a signal, such as a detectable label, present on one or both nucleic acid molecules (or antibody or protein as appropriate).

The binding between an oligomer and its target nucleic acid is frequently characterized by the temperature (Tₘ) at which 50% of the oligomer is melted from its target. A higher (Tₘ) means a stronger or more stable complex relative to a complex with a lower (Tₘ).

Biomarker:

Molecular, biological or physical attributes that characterize a physiological or cellular state and that can be objectively measured to detect or define disease progression or predict or quantify therapeutic responses. A biomarker is a characteristic that is objectively measured and evaluated as an indicator of normal biologic processes, pathogenic processes, or pharmacologic responses to a therapeutic intervention. A biomarker may be any molecular structure produced by a cell or organism. A biomarker may be expressed inside any cell or tissue; accessible on the surface of a tissue or cell; structurally inherent to a cell or tissue such as a structural component, secreted by a cell or tissue, produced by the breakdown of a cell or tissue through processes such as necrosis, apoptosis or the like; or any combination of these. A biomarker may be any protein, carbohydrate, fat, nucleic acid, catalytic site, or any combination of these such as an enzyme, glycoprotein, cell membrane, virus, cell, organ, organelle, or any uni- or multimolecular structure or any other such structure now known or yet to be disclosed whether alone or in combination.

A biomarker may be represented by the sequence of a nucleic acid from which it can be derived or any other chemical structure. Examples of such nucleic acids include miRNA, tRNA, siRNA, mRNA, cDNA, or genomic DNA sequences including any complimentary sequences thereof.

One example of a biomarker is a protein or RNA molecule expressed by a gene wherein expression of the protein or RNA signifies the presence of Alzheimer's disease. One further example is any expression product of the CCR6 gene.

cDNA (Complementary DNA):

A piece of DNA lacking internal, non-coding segments (introns) and regulatory sequences which determine transcription. cDNA can be synthesized by reverse transcription from messenger RNA (mRNA) extracted from cells, for example CCR6 cDNA reverse transcribed from CCR6 mRNA. The amount of CCR6 cDNA reverse transcribed from CCR6 mRNA can be used to determine the amount of CCR6 mRNA present in a biological sample and thus the amount of expression of CCR6.

CD19

(Cluster of Differentiation 19): A protein encoded by the CD19 gene. CD19 is expressed on follicular dendritic cells and B cells. It is present on B cells from earliest recognizable B-lineage cells during development to B-cell blasts but is lost on maturation to plasma cells. CD19 primarily acts as a B cell co-receptor in conjunction with CD21 and CD81. Upon activation, the cytoplasmic tail of CD19 becomes phosphorylated, which leads to binding by Src-family kinases and recruitment of PI-3 kinase. Exemplary amino acid sequences of CD19 can be found on GENBANK® at accession nos. AAA37388, AAA37390, AAA69966, AAD02340, BAB60954 and AAB60697, all of which are incorporated by reference as available Jun. 15, 2010.

Chemokine Receptor 6 (CCR6):

CCR6 also known as CD196 is an integral membrane protein that specifically binds and respond to chemokine ligand 20 (CCL20) (also known as liver activation regulated chemokine (LARC) or Macrophage Inflammatory Protein-3 (MIP3A)). CCR6 is in the family of G protein-linked receptors known as seven transmembrane (7-TM) proteins, because they span the cell membrane seven times. CCR6 is the receptor for chemokine ligand 20 (CCL20) (also known as liver activation regulated chemokine (LARC) or Macrophage Inflammatory Protein-3 (MIP3A)). CCR6 has been shown to be involved in B-lineage maturation and antigen-driven B-cell differentiation, and it may regulate the migration and recruitment of dentritic and T cells during inflammatory and immunological responses. Representative nucleic acid and protein sequences of CCR6 are included as SEQ ID NO: 1, SEQ ID NO: 2, SEQ ID NO: 3, and SEQ ID NO: 4.

Contacting:

Placement in direct physical association; includes solid, liquid, and gaseous associations. Contacting includes contact between one molecule and another molecule. Contacting can occur in vitro with isolated cells or tissue or in vivo by administering to a subject, such as the administration of a treatment for Alzheimer's disease to a subject. The concept of contacting may also be encompassed by adding a molecule to a solid, liquid, or gaseous mixture.

Control:

A reference standard. A control can be a known value indicative of basal expression of a gene, for example the amount of CCR6 expressed in mononuclear cells from peripheral blood and/or lymphoid tissue in a subject that does not have Alzheimer's disease or a predisposition for developing Alzheimer's disease. A difference between the expression in a test sample (such as a biological sample obtained from a subject) and a control can be an increase or conversely a decrease.

Cytokine:

The term “cytokine” is used as a generic name for a diverse group of soluble proteins and peptides that act as humoral regulators at nano- to picomolar concentrations and which, either under normal or pathological conditions, modulate the functional activities of individual cells and tissues. These proteins also mediate interactions between cells directly and regulate processes taking place in the extracellular environment. Many cytokines act as cellular survival factors by preventing programmed cell death. Cytokines include both naturally occurring peptides and variants that retain full or partial biological activity.

Determining Expression, Such as Detecting Expression of a Gene Product:

Detection of a level of expression in either a qualitative or quantitative manner, for example by detecting nucleic acid or protein (such as a CCR6 nucleic acid or protein) by routine methods known in the art.

Diagnosis:

The process of identifying a disease (such as Alzheimer's disease) by its signs, symptoms and results of various tests, for example tests for the expression of CCR6. The conclusion reached through that process is also called “a diagnosis.”

Differential Expression or Altered Expression:

A difference, such as an increase or decrease, in the amount of messenger RNA, the conversion of mRNA to a protein, or both. In some examples, the difference is relative to a control or threshold level of expression, such as an amount of gene expression in tissue not affected by a disease or from a different subject who does not have Alzheimer's disease. Detecting differential expression can include measuring a change in gene or protein expression, such as a change in expression of CCR6.

DNA (Deoxyribonucleic Acid):

A long chain polymer which includes the genetic material of most living organisms (some viruses have genes including ribonucleic acid, RNA). The repeating units in DNA polymers are four different nucleotides, each of which includes one of the four bases, adenine, guanine, cytosine and thymine bound to a deoxyribose sugar to which a phosphate group is attached. Triplets of nucleotides, referred to as codons, in DNA molecules code for amino acid in a polypeptide. The term codon is also used for the corresponding (and complementary) sequences of three nucleotides in the mRNA into which the DNA sequence is transcribed.

Decreased Expression, Downregulated, or Inactivation:

When used in reference to the expression of a nucleic acid molecule, such as a gene, refers to any process which results in a decrease in production of a gene product. A gene product can be RNA (such as mRNA, rRNA, tRNA, and structural RNA) or protein. Therefore, gene downregulation or deactivation includes processes that decrease transcription of a gene or translation of mRNA.

Examples of processes that decrease transcription include those that facilitate degradation of a transcription initiation complex, those that decrease transcription initiation rate, those that decrease transcription elongation rate, those that decrease processivity of transcription and those that increase transcriptional repression. Gene downregulation can include reduction of expression above an existing level. Examples of processes that decrease translation include those that decrease translational initiation, those that decrease translational elongation and those that decrease mRNA stability.

Gene downregulation includes any detectable decrease in the production of a gene product. In certain examples, production of a gene product decreases by at least 2-fold, for example at least 3-fold or at least 4-fold, as compared to a control (such an amount of gene expression in a sample obtained from a subject who does not have Alzheimer's disease or a predisposition for developing Alzheimer's disease, or a standard value indicative of basal expression of a gene such as CCR6).

Expression:

The process by which the coded information of a gene is converted into an operational, non-operational, or structural part of a cell, such as the synthesis of a protein. Gene expression can be influenced by external signals. For instance, exposure of a cell to a hormone may stimulate expression of a hormone-induced gene. Different types of cells can respond differently to an identical signal. Expression of a gene also can be regulated anywhere in the pathway from DNA to RNA to protein. Regulation can include controls on transcription, translation, RNA transport and processing, degradation of intermediary molecules such as mRNA, or through activation, inactivation, compartmentalization or degradation of specific protein molecules after they are produced.

Laboratory standards and values may be set based on a known or determined population value (for example, a value representing expression of a gene for a particular parameter, such as expression of a gene that encodes CCR6) and can be supplied in the format of a graph or table that permits comparison of measured, experimentally determined values.

Hybridization:

To form base pairs between complementary regions of two strands of DNA, RNA, or between DNA and RNA (such as CCR6 RNA and/or DNA), thereby forming a duplex molecule. Hybridization conditions resulting in particular degrees of stringency will vary depending upon the nature of the hybridization method and the composition and length of the hybridizing nucleic acid sequences. Generally, the temperature of hybridization and the ionic strength (such as the Na⁺ concentration) of the hybridization buffer will determine the stringency of hybridization. Calculations regarding hybridization conditions for attaining particular degrees of stringency are discussed in Sambrook et al., (1989) Molecular Cloning, second edition, Cold Spring Harbor Laboratory, Plainview, N.Y. (chapters 9 and 11). The following is an exemplary set of hybridization conditions and is not limiting:

Very High Stringency (Detects Sequences that Share at Least 90% Identity)

Hybridization: 5×SSC at 65° C. for 16 hours

Wash twice: 2×SSC at room temperature (RT) for 15 minutes each

Wash twice: 0.5×SSC at 65° C. for 20 minutes each

High Stringency (Detects Sequences that Share at Least 80% Identity)

Hybridization: 5×-6×SSC at 65° C.-70° C. for 16-20 hours

Wash twice: 2×SSC at RT for 5-20 minutes each

Wash twice: 1×SSC at 55° C.-70° C. for 30 minutes each

Low Stringency (Detects Sequences that Share at Least 50% Identity)

Hybridization: 6×SSC at RT to 55° C. for 16-20 hours

Wash at least twice: 2×-3×SSC at RT to 55° C. for 20-30 minutes each.

Inhibiting or Treating a Disease:

Inhibiting the full development of a disease or condition, for example, in a subject who is at risk for a disease such as Alzheimer's disease. “Treatment” refers to a therapeutic intervention that ameliorates a sign or symptom of a disease or pathological condition after it has begun to develop, whether or not the subject has developed symptoms of the disease. The term “ameliorating,” with reference to a disease, pathological condition or symptom, refers to any observable beneficial effect of the treatment. The beneficial effect can be evidenced, for example, by a delayed onset of clinical symptoms of the disease in a susceptible subject, a reduction in severity of some or all clinical symptoms of the disease, a slower progression of the disease, a reduction in the number of relapses of the disease, an improvement in the memory and/or cognitive function of the subject, or by other parameters well known in the art that are specific to Alzheimer's disease.

Increase Expression, Upregulated or Activation:

When used in reference to the expression of a nucleic acid molecule, such as a gene, refers to any process which results in an increase in production of a gene product. A gene product can be RNA (such as mRNA, rRNA, tRNA, and structural RNA) or protein. Therefore, gene upregulation or activation includes processes that increase transcription of a gene or translation of mRNA.

Examples of processes that increase transcription include those that facilitate formation of a transcription initiation complex, those that increase transcription initiation rate, those that increase transcription elongation rate, those that increase processivity of transcription and those that relieve transcriptional repression (for example by blocking the binding of a transcriptional repressor). Gene upregulation can include inhibition of repression as well as stimulation of expression above an existing level. Examples of processes that increase translation include those that increase translational initiation, those that increase translational elongation and those that increase mRNA stability.

Gene upregulation includes any detectable increase in the production of a gene product. In certain examples, production of a gene product increases by at least 2-fold, for example at least 3-fold or at least 4-fold, as compared to a control (such an amount of gene expression in a sample obtained from a subject who does not have Alzheimer's disease or a predisposition for developing Alzheimer's disease, or a standard value indicative of basal expression of a gene, such as CCR6).

Label:

A detectable compound or composition that is conjugated directly or indirectly to another molecule to facilitate detection of that molecule. Specific, non-limiting examples of labels include fluorescent tags, enzymatic linkages, and radioactive isotopes. In some examples, a label is attached to an antibody or nucleic acid to facilitate detection of the molecule that the antibody or nucleic acid specifically binds, such as a CCR6 protein or nucleic acid.

Leukocyte:

Cells in the blood, also termed “white cells,” that are involved in defending the body against infective organisms and foreign substances. Leukocytes are produced in the bone marrow. There are 5 main types of white blood cell, subdivided between 2 main groups: polymorphonuclear leukocytes (neutrophils, eosinophils, basophils) and mononuclear leukocytes (monocytes and lymphocytes). Mononuclear leukocytes may also be termed mononuclear cells and the terms may be used interchangeably.

Lymphocytes:

A type of white blood cell that is involved in the immune defenses of the body. There are two main types of lymphocytes: B cells and T cells. T cells are white blood cells critical to the immune response. T cells include, but are not limited to, CD4⁺ T cells and CD8⁺ T cells. A CD4⁺ T lymphocyte is an immune cell that carries a marker on its surface known as “cluster of differentiation 4” (CD4). These cells, also known as helper T cells, help orchestrate the immune response, including antibody responses as well as killer T cell responses. CD8⁺ T cells carry the “cluster of differentiation 8” (CD8) marker. B cells are white blood cells critical to the antibody response. B cells mature within the bone marrow and leave the marrow expressing an antigen binding antibody on their cell surface. When a naïve B cell encounters the antigen for which its membrane-bound antibody is specific, the cell begins to divide rapidly and its progeny differentiate into memory B cells and effector cells termed “plasma cells.” Memory B cells have a longer life span and continue to express membrane-bound antibody with the same specificity as the original parent cell. Plasma cells do not produce membrane-bound antibody but instead produce the antibody in a form that can be secreted. Secreted antibodies are the major effector of humoral immunity.

Mass Spectrometry:

A method wherein, a sample is analyzed by generating gas phase ions from the sample, which are then separated according to their mass-to-charge ratio (m/z) and detected. Methods of generating gas phase ions from a sample include electrospray ionization (ESI), matrix-assisted laser desorption-ionization (MALDI), surface-enhanced laser desorption-ionization (SELDI), chemical ionization, and electron-impact ionization (EI). Separation of ions according to their m/z ratio can be accomplished with any type of mass analyzer, including quadrupole mass analyzers (Q), time-of-flight (TOF) mass analyzers, magnetic sector mass analyzers, 3D and linear ion traps (IT), Fourier-transform ion cyclotron resonance (FT-ICR) analyzers, and combinations thereof (for example, a quadrupole-time-of-flight analyzer, or Q-TOF analyzer). Prior to separation, the sample may be subjected to one or more dimensions of chromatographic separation, for example, one or more dimensions of liquid or size exclusion chromatography or gel-electrophoretic separation.

Nucleic Acid Molecules Representing Genes:

Any nucleic acid, for example DNA (intron or exon or both), cDNA, or RNA (such as mRNA), of any length suitable for use as a probe or other indicator molecule, and that is informative about the corresponding gene.

Nucleic Acid Molecules:

A deoxyribonucleotide or ribonucleotide polymer including, without limitation, cDNA, mRNA, genomic DNA, and synthetic (such as chemically synthesized) DNA. The nucleic acid molecule can be double-stranded or single-stranded. Where single-stranded, the nucleic acid molecule can be the sense strand or the antisense strand. In addition, nucleic acid molecule can be circular or linear.

Oligonucleotide:

A plurality of joined nucleotides joined by native phosphodiester bonds, between about 6 and about 300 nucleotides in length. An oligonucleotide analog refers to moieties that function similarly to oligonucleotides but have non-naturally occurring portions. For example, oligonucleotide analogs can contain non-naturally occurring portions, such as altered sugar moieties or inter-sugar linkages, such as a phosphorothioate oligodeoxynucleotide.

Particular oligonucleotides and oligonucleotide analogs can include linear sequences up to about 200 nucleotides in length, for example a sequence (such as DNA or RNA) that is at least 6 nucleotides, for example at least 8, at least 10, at least 15, at least 20, at least 21, at least 25, at least 30, at least 35, at least 40, at least 45, at least 50, at least 100 or even at least 200 nucleotides long, or from about 6 to about 50 nucleotides, for example about 10-25 nucleotides, such as 12, 15 or 20 nucleotides.

An oligonucleotide probe is a short sequence of nucleotides, such as at least 8, at least 10, at least 15, at least 20, at least 21, at least 25, or at least 30 nucleotides in length, used to detect the presence of a complementary sequence by molecular hybridization. In particular examples, oligonucleotide probes include a label that permits detection of oligonucleotide probe:target sequence hybridization complexes.

Peptide:

Any compound composed of amino acids or amino acid analogs chemically bound together. Peptide as used herein includes oligomers of amino acids, amino acid analog, or small and large peptides, including polypeptides or proteins. Any chain of amino acids, regardless of length or post-translational modification (such as glycosylation or phosphorylation). In one example, a peptide is a CCR6 protein or fragment thereof.

A polypeptide is a polymer in which the monomers are amino acid residues which are joined together through amide bonds. When the amino acids are alpha-amino acids, either the L-optical isomer or the D-optical isomer can be used. The terms “polypeptide” or “protein” as used herein are intended to encompass any amino acid sequence and include modified sequences such as glycoproteins. The term “polypeptide” is specifically intended to cover naturally occurring proteins, as well as those which are recombinantly or synthetically produced. The term “residue” or “amino acid residue” includes reference to an amino acid that is incorporated into a protein, polypeptide, or peptide.

Probes and Primers:

A probe comprises an isolated nucleic acid capable of hybridizing to a target nucleic acid (such as a CCR6 nucleic acid molecule). A detectable label or reporter molecule can be attached to a probe. Typical labels include radioactive isotopes, enzyme substrates, co-factors, ligands, chemiluminescent or fluorescent agents, haptens, and enzymes. Methods for preparing and using nucleic acid probes and primers are described, for example, in Sambrook et al. (In Molecular Cloning: A Laboratory Manual, CSHL, New York, 1989), Ausubel et al. (ed.) (In Current Protocols in Molecular Biology, John Wiley & Sons, New York, 1998), and Innis et al. (PCR Protocols, A Guide to Methods and Applications, Academic Press, Inc., San Diego, Calif., 1990). Methods for labeling and guidance in the choice of labels appropriate for various purposes are discussed, for example in Sambrook et al. (In Molecular Cloning: A Laboratory Manual, CSHL, New York, 1989) and Ausubel et al. (In Current Protocols in Molecular Biology, John Wiley & Sons, New York, 1998).

In a particular example, a probe includes at least one fluorophore, such as an acceptor fluorophore or donor fluorophore. For example, a fluorophore can be attached at the 5′- or 3′-end of the probe. In specific examples, the fluorophore is attached to the base at the 5′-end of the probe, the base at its 3′-end, the phosphate group at its 5′-end or a modified base, such as a T internal to the probe.

Probes are generally at least 12 nucleotides in length, such as at least 12, at least 13, at least 14, at least 15, at least 16, at least 17, at least 18, at least 19, least 20, at least 21, at least 22, at least 23, at least 24, at least 25, at least 26, at least 27, at least 28, at least 29, at least 30, or more contiguous nucleotides complementary to the target nucleic acid molecule, such as 12-30 nucleotides, 15-30 nucleotides, 20-30 nucleotides, or 12-29 nucleotides.

Primers are short nucleic acid molecules, for instance DNA oligonucleotides 10 nucleotides or more in length, which can be annealed to a complementary target nucleic acid molecule by nucleic acid hybridization to form a hybrid between the primer and the target nucleic acid strand. A primer can be extended along the target nucleic acid molecule by a polymerase enzyme. Therefore, primers can be used to amplify a target nucleic acid molecule (such as a portion of a CCR6 nucleic acid molecule).

The specificity of a primer increases with its length. Thus, for example, a primer that includes 30 consecutive nucleotides will anneal to a target sequence with a higher specificity than a corresponding primer of only 15 nucleotides. Thus, to obtain greater specificity, probes and primers can be selected that include at least 15, 20, 25, 30, 35, 40, 45, 50 or more consecutive nucleotides. In particular examples, a primer is at least 15 nucleotides in length, such as at least 15 contiguous nucleotides complementary to a target nucleic acid molecule. Particular lengths of primers that can be used to practice the methods of the present disclosure (for example, to amplify a region of a CCR6 nucleic acid molecule) include primers having at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 45, at least 50, or more contiguous nucleotides complementary to the target nucleic acid molecule to be amplified, such as a primer of 15-50 nucleotides, 20-50 nucleotides, or 15-30 nucleotides.

Primer pairs can be used for amplification of a nucleic acid sequence, for example, by PCR, real-time PCR, or other nucleic-acid amplification methods known in the art. An “upstream” or “forward” primer is a primer 5′ to a reference point on a nucleic acid sequence. A “downstream” or “reverse” primer is a primer 3′ to a reference point on a nucleic acid sequence. In general, at least one forward and one reverse primer are included in an amplification reaction.

Nucleic acid probes and/or primers can be readily prepared based on the nucleic acid molecules provided herein. PCR primer pairs and probes can be derived from a known sequence (such as the CCR6 nucleic acid molecules as set forth in SEQ ID NO: 1, and/or SEQ ID NO: 3) for example, by using computer programs intended for that purpose such as Primer (Version 0.5, © 1991, Whitehead Institute for Biomedical Research, Cambridge, Mass.) or PRIMER EXPRESS® Software (Applied Biosystems, AB, Foster City, Calif.).

Examples of nucleic acid probes and primers that may be used in the nucleic acid amplification of all or part of a CCR6 nucleic acid molecule (such as SEQ ID NO: 1 or SEQ ID NO: 3 are included herein as Primer Sets and Primer/Probe Sets.

Pharmaceutical Composition:

A chemical compound or composition capable of inducing a desired therapeutic or prophylactic effect when properly administered to a subject, for example a subject with Alzheimer's disease or a predisposition to developing Alzheimer's disease. A pharmaceutical composition can include a therapeutic agent, a diagnostic agent or a pharmaceutical agent. A therapeutic or pharmaceutical agent is one that alone or together with an additional compound induces the desired response (such as inducing a therapeutic or prophylactic effect when administered to a subject). In a particular example, a pharmaceutical agent is an agent that significantly reduces one or more symptoms associated with Alzheimer's disease. A pharmaceutical composition may be a member of a group of compounds. Pharmaceutical compositions may be grouped by any characteristic including chemical structure and the molecular target they affect.

Pharmaceutically Acceptable Carriers or Vehicles:

The pharmaceutically acceptable carriers (vehicles) useful in this disclosure are conventional. Remington's Pharmaceutical Sciences, by E. W. Martin, Mack Publishing Co., Easton, Pa., 15th Edition (1975), describes compositions and formulations suitable for pharmaceutical delivery of one or more therapeutic compounds or molecules, such as the treatments for Alzheimer's disease described herein. In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. In a particular embodiment the carrier is one that allows the therapeutic compound to cross the blood-brain barrier. For solid compositions (for example, powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Prognosis:

A prediction of the course of a disease, such as Alzheimer's disease. The prediction can include determining the likelihood of a subject to develop the disease, to respond to a particular therapy (for example an Alzheimer's therapy), or combinations thereof.

Sample (or Biological Sample):

A biological specimen containing genomic DNA, RNA (including mRNA), protein, or combinations thereof, that is obtained from a subject. Examples include, but are not limited to, peripheral blood, lymphoid tissue (such as spleen tissue) urine, saliva, tissue biopsy, needle aspirates, surgical specimen, and autopsy material. In one example, a sample includes peripheral blood obtained from a subject, with or without Alzheimer's disease. In one example, a sample includes lymphatic tissue, such as spleen tissue, obtained from a subject, with or without Alzheimer's disease. In some examples, a biological sample does not include neurological tissue, such as brain tissue.

Obtaining a biological sample from a subject includes, but need not be limited to any method of collecting a particular sample known in the art. Obtaining a biological sample from a subject also encompasses receiving a sample that was collected at a different location than where a method is performed; receiving a sample that was collected by a different individual than an individual that performs the method, receiving a sample that was collected at any time period prior to the performance of the method, receiving a sample that was collected using a different instrument than the instrument that performs the method, or any combination of these. Obtaining a biological sample from a subject also encompasses situations in which the collection of the sample and performance of the method are performed at the same location, by the same individual, at the same time, using the same instrument, or any combination of these.

A biological sample encompasses any fraction of a biological sample or any component of a biological sample that may be isolated and/or purified from the biological sample. For example: when cells are isolated from blood or tissue, including specific cell types sorted on the basis of biomarker expression; or when nucleic acid or protein is purified from a fluid or tissue; or when blood is separated into fractions such as plasma, serum, buffy coat PBMC's or other cellular and non-cellular fractions on the basis of centrifugation and/or filtration. A biological sample further encompasses biological samples or fractions or components thereof that have undergone a transformation of mater or any other manipulation. For example, a cDNA molecule made from reverse transcription of mRNA purified from a biological sample may be termed a bi0logical sample.

A biological sample from a subject may be identified as comprising mononuclear cells. Mononuclear cells are often isolated from whole blood through of whole blood over Ficoll®, a branched polysachharide. After centrifugation over Ficoll®, the mononuclear cells form a “buffy coat” beneath the plasma layer. Examples of mononuclear cells include lymphocytes (such as B and T cells), monocytes, macrophages, and dendritic cells. While blood is an efficient source of mononuclear cells, mononuclear cells may be obtained from almost any tissue type, including tissues undergoing inflammatory or other immune responses. As a result, a biological sample that comprises mononuclear cells includes any tissue from which mononuclear cells may be isolated or purified, such as whole blood, spleen, lymph nodes, or any tissue that is the site of immune system activity. A biological sample that comprises mononuclear cells also encompasses any sorted population of mononuclear cells such as CD4+ T-cells, CD8+ T-cells, CD19+ B cells, or T-cells, B-cells, monocytes, macrophages, and dendritic cells generally.

Sequence Identity/Similarity:

The identity/similarity between two or more nucleic acid sequences, or two or more amino acid sequences, is expressed in terms of the identity or similarity between the sequences. Sequence identity can be measured in terms of percentage identity; the higher the percentage, the more identical the sequences are. Sequence similarity can be measured in terms of percentage similarity (which takes into account conservative amino acid substitutions); the higher the percentage, the more similar the sequences are.

Methods of alignment of sequences for comparison are well known in the art. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al. Computer Appls. in the Biosciences 8, 155-65, 1992; and Pearson et al., Meth. Mol. Bio. 24:307-31, 1994. Altschul et al., J. Mol. Biol. 215:403-10, 1990, presents a detailed consideration of sequence alignment methods and homology calculations.

The NCBI Basic Local Alignment Search Tool (BLAST) (Altschul et al., J. Mol. Biol. 215:403-10, 1990) is available from several sources, including the National Center for Biological Information (NCBI, National Library of Medicine, Building 38A, Room 8N805, Bethesda, Md. 20894) and on the Internet, for use in connection with the sequence analysis programs blastp, blastn, blastx, tblastn and tblastx. Additional information can be found at the NCBI web site.

BLASTN is used to compare nucleic acid sequences, while BLASTP is used to compare amino acid sequences. If the two compared sequences share homology, then the designated output file will present those regions of homology as aligned sequences. If the two compared sequences do not share homology, then the designated output file will not present aligned sequences.

Once aligned, the number of matches is determined by counting the number of positions where an identical nucleotide or amino acid residue is presented in both sequences. The percent sequence identity is determined by dividing the number of matches either by the length of the sequence set forth in the identified sequence, or by an articulated length (such as 100 consecutive nucleotides or amino acid residues from a sequence set forth in an identified sequence), followed by multiplying the resulting value by 100. For example, a nucleic acid sequence that has 1166 matches when aligned with a test sequence having 1154 nucleotides is 75.0 percent identical to the test sequence (1166÷1554*100=75.0). The percent sequence identity value is rounded to the nearest tenth. For example, 75.11, 75.12, 75.13, and 75.14 are rounded down to 75.1, while 75.15, 75.16, 75.17, 75.18, and 75.19 are rounded up to 75.2. The length value will always be an integer. In another example, a target sequence containing a 20-nucleotide region that aligns with 20 consecutive nucleotides from an identified sequence as follows contains a region that shares 75 percent sequence identity to that identified sequence (that is, 15÷20*100=75).

For comparisons of amino acid sequences of greater than about 30 amino acids, the Blast 2 sequences function is employed using the default BLOSUM62 matrix set to default parameters, (gap existence cost of 11, and a per residue gap cost of 1). Homologs are typically characterized by possession of at least 70% sequence identity counted over the full-length alignment with an amino acid sequence using the NCBI Basic Blast 2.0, gapped blastp with databases such as the nr or swissprot database. Queries searched with the blastn program are filtered with DUST (Hancock and Armstrong, 1994, Comput. Appl. Biosci. 10:67-70). Other programs use SEG. In addition, a manual alignment can be performed. Proteins with even greater similarity will show increasing percentage identities when assessed by this method, such as at least about 75%, 80%, 85%, 90%, 95%, 98%, or 99% sequence identity to a protein according to SEQ ID NO: 2 or SEQ ID NO: 4.

When aligning short peptides (fewer than around 30 amino acids), the alignment is be performed using the Blast 2 sequences function, employing the PAM30 matrix set to default parameters (open gap 9, extension gap 1 penalties). Proteins with even greater similarity to the reference sequence will show increasing percentage identities when assessed by this method, such as at least about 60%, 70%, 75%, 80%, 85%, 90%, 95%, 98%, or 99% sequence identity to a protein according to SEQ ID NO: 2 or SEQ ID NO: 4. When less than the entire sequence is being compared for sequence identity, homologs will typically possess at least 75% sequence identity over short windows of 10-20 amino acids, and can possess sequence identities of at least 85%, 90%, 95% or 98% depending on their identity to the reference sequence. Methods for determining sequence identity over such short windows are described at the NCBI web site.

One indication that two nucleic acid molecules are closely related is that the two molecules hybridize to each other under stringent conditions, as described above. Nucleic acid sequences that do not show a high degree of identity may nevertheless encode identical or similar (conserved) amino acid sequences, due to the degeneracy of the genetic code. Changes in a nucleic acid sequence can be made using this degeneracy to produce multiple nucleic acid molecules that all encode substantially the same protein. Such homologous nucleic acid sequences can, for example, possess at least about 60%, 70%, 80%, 90%, 95%, 98%, or 99% sequence identity to a nucleic acid that encodes a protein according to SEQ ID NO: 1 or SEQ ID NO: 3 can be determined by this method.

Specific Binding Agent:

An agent that binds substantially or preferentially only to a defined target such as a protein, enzyme, polysaccharide, oligonucleotide, DNA, RNA, recombinant vector or a small molecule. In an example, a “specific binding agent” is capable of binding to a CCR6 gene product, such as a CCR6 mRNA, cDNA, or protein. Thus, a nucleic acid-specific binding agent binds substantially only to the defined nucleic acid, such as RNA, or to a specific region within the nucleic acid.

A protein-specific binding agent binds substantially only the defined protein, or to a specific region within the protein. For example, a “specific binding agent” includes antibodies and other agents that bind substantially to a specified polypeptide, for example a specific binding agent that specifically binds CCR6, can be an antibody, for example a monoclonal or ploy clonal antibody or a ligand for CCR6, such as CCL20 (SEQ ID NO: 31). Antibodies can be monoclonal or polyclonal antibodies that are specific for the polypeptide, such as CCR6, as well as immunologically effective portions (“fragments”) thereof. The determination that a particular agent binds substantially only to a specific polypeptide may readily be made by using or adapting routine procedures. One suitable in vitro assay makes use of the Western blotting procedure (described in many standard texts, including Harlow and Lane, Using Antibodies: A Laboratory Manual, CSHL, New York, 1999).

A specific binding agent that binds to a particular biomarker may also be called a reagent that specifically binds a biomarker. These terms may be used interchangeably.

Standard:

A substance or solution of a substance of known amount, purity or concentration. A standard can be compared (such as by spectrometric, chromatographic, or spectrophotometric analysis) to an unknown sample (of the same or similar substance) to determine the presence of the substance in the sample and/or determine the amount, purity or concentration of the unknown sample. In one embodiment, a standard is a peptide standard. An internal standard is a compound that is added in a known amount to a sample prior to sample preparation and/or analysis and serves as a reference for calculating the concentrations of the components of the sample. In one example, nucleic acid standards serve as reference values for expression levels of specific nucleic acids, such as CCR6 nucleic acids. In some examples, peptide standards serve as reference values for expression levels of specific peptides, such as CCR6 proteins. Isotopically-labeled peptides are particularly useful as internal standards for peptide analysis since the chemical properties of the labeled peptide standards are almost identical to their non-labeled counterparts. Thus, during chemical sample preparation steps (such as chromatography, for example, HPLC) any loss of the non-labeled peptides is reflected in a similar loss of the labeled peptides.

Subject:

Living multi-cellular vertebrate organisms, a category that includes human and non-human mammals, such as mice. In some examples a subject is a male. In some examples a subject is a female.

Symptom and Sign:

Any subjective evidence of disease or of a subject's condition, for example, such evidence as perceived by the subject; a noticeable change in a subject's condition indicative of some bodily or mental state. A sign may be any abnormality indicative of disease, discoverable on examination or assessment of a subject. A sign is generally an objective indication of disease.

Therapeutically Effective Amount or Concentration:

An amount of a composition that alone, or together with an additional therapeutic agent(s) sufficient to achieve a desired effect in a subject, or in a cell, being treated with the agent. The effective amount of the agent will be dependent on several factors, including, but not limited to the subject or cells being treated, and the manner of administration of the therapeutic composition. In one example, a therapeutically effective amount or concentration is one that is sufficient to prevent advancement, delay progression, or to cause regression of a disease, or which is capable of reducing symptoms caused by the disease, such as such as Alzheimer's disease.

In one example, a desired response is to reduce or inhibit one or more symptoms associated with Alzheimer's disease. The one or more symptoms do not have to be completely eliminated for the composition to be effective. For example, a composition can decrease the sign or symptom by a desired amount, for example by at least 20%, at least 50%, at least 80%, at least 90%, at least 95%, at least 98%, or even at least 100%, as compared to the sign or symptom in the absence of the composition.

A therapeutically effective amount of a disclosed pharmaceutical composition can be administered in a single dose, or in several doses, for example daily, during a course of treatment. However, the therapeutically effective amount can depend on the subject being treated, the severity and type of the condition being treated, and the manner of administration. For example, a therapeutically effective amount of such agent can vary from about 100 μg-10 mg per kg body weight if administered intravenously.

Tissue:

A plurality of functionally related cells. A tissue can be a suspension, a semi-solid, or solid. Tissue includes cells collected from a subject, such as the spleen or a portion thereof.

Wild-Type (WT):

A subject not affected with a specific disease or disorder, such as a subject not affected with Alzheimer's disease.

Unless otherwise explained, all technical and scientific terms used herein have the same meaning as commonly understood by one of ordinary skill in the art to which a disclosed invention belongs. The singular terms “a,” “an,” and “the” include plural referents unless context clearly indicates otherwise. Similarly, the word “or” is intended to include “and” unless the context clearly indicates otherwise. “Comprising” means “including.” Hence “comprising A or B” means “including A” or “including B” or “including A and B.

Suitable methods and materials for the practice and/or testing of embodiments of a disclosed invention are described below. Such methods and materials are illustrative only and are not intended to be limiting. Other methods and materials similar or equivalent to those described herein can be used. For example, conventional methods well known in the art to which a disclosed invention pertains are described in various general and more specific references, including, for example, Sambrook et al., Molecular Cloning: A Laboratory Manual, 2d ed., Cold Spring Harbor Laboratory Press, 1989; Sambrook et al., Molecular Cloning: A Laboratory Manual, 3d ed., Cold Spring Harbor Press, 2001; Ausubel et al., Current Protocols in Molecular Biology, Greene Publishing Associates, 1992 (and Supplements to 2000); Ausubel et al., Short Protocols in Molecular Biology: A Compendium of Methods from Current Protocols in Molecular Biology, 4th ed., Wiley & Sons, 1999; Harlow and Lane, Antibodies: A Laboratory Manual, Cold Spring Harbor Laboratory Press, 1990; and Harlow and Lane, Using Antibodies: A Laboratory Manual, Cold Spring Harbor Laboratory Press, 1999.

Additional terms commonly used in molecular genetics can be found in Benjamin Lewin, Genes V published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

All sequences associated with the GENBANK® Accession Nos. mentioned herein are incorporated by reference in their entirety as were present on Jun. 5, 2010, to the extent permissible by applicable rules and/or law.

### II. Description of Several Embodiments

The inflammatory status of the brain in subjects as well as animal models of Alzheimer's disease (AD) has been studied. Accumulation of activated microglia producing TNF-α and MCP-1 contribute to the pathology of the disease. However, prior to this disclosure, little was known about the changes in the spleen and associated peripheral immunity that might contribute to AD pathology.

To investigate and characterize the phenotypic and functional changes that occur in mononuclear cells in the spleen, blood and brain, a triple transgenic (3×Tg-AD) mouse model was chosen for initial examinations. The goal of this investigation was the identification of Alzheimer's disease associated biomarkers useful in the diagnosis of Alzheimer's disease. To this end, the expression of biomarkers related to inflammation was determined in young presymptomatic and older symptomatic 3×Tg-AD. As disclosed herein, brain tissue from older symptomatic 3×Tg-AD female mice exhibited highly elevated mRNA expression of CCR6 compared to WT littermates without AD. Also disclosed herein is the discovery that there is an increase in the expression of CD19+CCR6+ cells in spleen from 3×Tg-AD female mice compared to WT littermates. Furthermore, CD19+CCR6+ B-cells are also increased in blood from 3×Tg-AD male mice compared to their WT counterparts.

Based upon the finding using the 3×Tg-AD mice, CCR6 expression was determined in mononuclear cells isolated from human subjects. CCR6 expression in subjects diagnosed with AD by clinical methods was elevated relative to CCR6 expression in age- and gender-matched healthy control human subjects.

As disclosed herein, for the first time it is demonstrated that the expression of CCR6 precedes the onset of clinical AD. Through monitoring the expression of CCR6 in peripheral blood and/or lymphoid tissue such as the spleen, it is possible to make a diagnosis of Alzheimer's disease prior to any clinically evident presentation of the disease in the subject. The ability of the present disclosure to predict Alzheimer's disease in a subject in the absence of clinical symptoms should prove invaluable to early intervention to reverse, halt or slow the progression of Alzheimer's disease and its debilitating consequences.

As disclosed herein, for the first time it is demonstrated that the expression of CCR6 in the peripheral blood may be used to differentiate AD patients from non-AD patients in humans. This represents an important peripheral blood biomarker that will be invaluable for the study, diagnosis, and treatment of AD.

A. Methods for Diagnosing or Predicting a Predisposition to Develop Alzheimer's Disease

Disclosed are methods for diagnosing or predicting a predisposition to develop Alzheimer's disease in a subject, such as a human subject. In some embodiments, the methods include obtaining a biological sample from the subject, for example a sample of peripheral blood cells and/or lymphoid tissue, such as spleen tissue. In some examples the biological sample includes B-cells, such as CD19 positive B-cells. The amount of chemokine receptor 6 (CCR6) expressed in the biological sample (for example a sample of peripheral blood cells or any fraction thereof, such as mononuclear cells, B-cells or any fraction thereof such as CD19 positive B-cells and/or lymphoid tissue, such as spleen tissue or lymph node tissue) is detected and compared to a control. The control may be indicative of a similar sample obtained from a subject who does not have Alzheimer's disease and does not have any predisposition for developing Alzheimer's disease. Alternatively, the reference value may be indicative of basal expression of CCR6 in the absence of Alzheimer's disease. The reference value may also be a threshold level of expression such that a level of CCR6 expression in a sample that exceeds the threshold value indicates that the subject from which the sample was obtained has AD. The control may be a sample from a subject collected earlier in time. relative to the control (such as an amount of CCR6 expressed in a normal biological sample, for example a reference value or range of values representing the expected CCR6 expressed levels in a normal lymphoid tissue or peripheral blood, such as B-cells, for example CD19 positive B-cells), the subject is diagnosed with AD and/or will develop AD sometime in the future. For example, expression at least 10%, at least 20%, at least 30%, at least 50%, at least 75%, at least 80%, at least 90%, at least 100%, at least 200% or even at least 500%, higher than the control, indicates that the subject (such as a human subject) has Alzheimer's disease and/or will go on to develop symptoms of AD sometime in the future. Development of symptoms may follow elevated expression of CCR6 by one month, three months, six months, one year, two years, five years, or ten years or more.

Conversely, a lesser value or maintenance of CCR6 expression in the biological sample (such as an amount of CCR6 expressed in a normal biological sample relative to the control indicates that the subject does not have and/or will not go on to develop symptoms of AD sometime in the future.

In some embodiments, the disclosed methods are used to determine if a subject has Alzheimer's disease, for example as a primary diagnosis of Alzheimer's disease or alternatively to confirm a diagnosis of Alzheimer's disease made be another method such as Magnetic Resonance Imagining (MRI) and/or an measurement of cognitive mental process made by a trained clinician.

The disclosed methods can also be used to select a subject for treatment for Alzheimer's disease. In such embodiments, a subject with an elevated CCR6 expressed in the biological sample relative to a control is one that would benefit from treatment for Alzheimer's disease and is thereby selected for treatment for Alzheimer's disease. For example, expression of CCR6 at least 10%, at least 20%, at least 30%, at least 50%, at least 75%, at least 80%, at least 90%, at least 100%, at least 200% or even at least 500%, relative to the control, indicates that the subject (such as a human subject) would benefit from treatment for Alzheimer's disease and is thereby selected for treatment for Alzheimer's disease. Conversely, repression or maintenance of CCR6 expression in the biological sample relative to the control indicates that the subject will not develop Alzheimer's disease and the subject would not be selected for treatment for Alzheimer's disease.

A test that determines CCR6 expression could be part of a regular physical examination and blood work, or could be conducted as a separate diagnostic test. The benefits of identifying subjects that have Alzheimer's disease prior to the development of symptoms are tremendous. For example, the subject may be treated for Alzheimer's disease prior to the development of symptoms of AD, including memory loss or loss of cognitive ability. Treatment regimens for AD may be more efficacious if they are administered prior to the onset of symptoms. In one embodiment, this assay is performed in a medical laboratory on a sample of peripheral blood, mononuclear cells isolated from the peripheral blood, serum or plasma or lymphoid tissue, such as spleen tissue.

Also disclosed are methods used in monitoring a subject's response, such as a human subject's response, to a treatment for Alzheimer's disease. In such methods, a first sample is obtained at a first time point and a second sample is obtained at second later time point from a single subject being treated for Alzheimer's disease. The first time point may occur prior to treatment and the second time point may occur following treatment. The expression level of CCR6 in the first sample may be compared to the expression level of CCR6 in the second sample. Either sample may be any sample that may be obtained from the subject, including but not limited to: whole blood, isolated peripheral blood cells, such as mononuclear cells or sorted B-cells, for example CD19 positive B-cells and/or lymphoid tissue, such as spleen or lymph node tissue.

Methods of monitoring a subject's response to treatment may also be used in the context of a clinical trial. A clinical trial may be any test of a new treatment, especially a clinical trial that tests a new treatment for AD. Clinical trials are often conducted according to the specifications of a governmental agency, ministry, or association, such as the Food and Drug Administration in the United States. The new treatment may be any new treatment including a new pharmaceutical composition never tested for any indication, tested for, but never approved for any indication, or a pharmaceutical composition that has been approved by the governmental agency for one or more indications other than AD. Additionally or alternatively, the treatment may comprise surgery, exercise or physical manipulation of the subject, herbal compositions, supplements, or any other therapeutic moiety.

A lower level of expression of CCR6 expressed in the second biological sample relative to the amount of CCR6 expressed in the first biological sample indicates that the subject is responding to the treatment for Alzheimer's disease. For example, a reduction of at least 10%, at least 20%, at least 30%, at least 50%, at least 75%, at least 80%, at least 90%, at least 95%, at least 98% or even at least 99%, indicates that the subject (such as a human subject with being treated for Alzheimer's disease) is responding to treatment. Conversely an increase in or maintenance of the amount of CCR6 expressed in the second biological sample relative to the amount of CCR6 expressed in the first biological sample indicates that the subject is not responding to the treatment for Alzheimer's disease. For example, an increase of at least 10%, at least 20%, at least 30%, at least 50%, at least 75%, at least 80%, at least 90%, at least 100%, at least 200% or even at least 500%, indicates that the subject (such as a human subject receiving a treatment for Alzheimer's disease) is not responding to treatment.

In some embodiments of the disclosed methods, a treatment for Alzheimer's disease is administered to the subject. Currently available treatments for Alzheimer's disease are known to those of ordinary skill and the art. Exemplary treatments for Alzheimer's disease include administration of cholinesterase inhibitors, neurotransmitters, non-steroidal anti-inflammatory agents, or any combination thereof. The invention also encompasses the use of experimental treatments not yet generally known in the art.

Cholinesterase inhibitors delay the breakdown of the neurotransmitter acetylcholine, a chemical in the brain that facilitates communication among nerve cells and is important for memory. Alzheimer's disease has been associated with inadequate levels of acetylcholine. While not being bound by theory, it is believed that cholinesterase inhibitors increase the levels of acetylcholine in the brain and thus improve memory in subjects affected with Alzheimer's disease. Examples of cholinesterase inhibitors for use in the treatment of Alzheimer's disease include galantamine (trade names RAZADYNE® and REMINYL®), rivastigmine (trade name EXELON®), donepezil (trade name ARICEPT®) and tacrine (trade name COGNEX®).

Neurotransmitters, such as memantine (trade name NAMENDA®) are believed to treat Alzheimer's disease by preventing brain cells from overexposure to another neurotransmitters called glutamate, excess levels of which contribute to the death of brain cells in subjects with Alzheimer's disease.

While not being bound by theory, non-steroidal anti-inflammatory agents (NSAIDs) are believe to treat Alzheimer's disease by interrupting the inflammatory process occurring in the brains of subjects affected by Alzheimer's disease. Examples of non-steroidal anti-inflammatory agents of use in treating Alzheimer's disease are: propionic acid derivatives, such as ibuprofen, naproxen, fenoprofen, ketoprofen, flurbiprofen, and oxaprozin; acidic acid derivative, such as indomethacin, sulindac, etodolac, and diclofenac; enolic acid derivatives, such as piroxicam, meloxicam, tenoxicam, droxicam, lornoxicam, and isoxicam; fenamic acid derivatives, such as mefenamic acid, meclofenamic acid, flufenamic acid, and tolfenamic acid; and COX-2 inhibitors, such as celecoxib, rofecoxib, valdecoxib, parecoxib, lumiracoxib, and etoricoxib; or any combination thereof.

The administration of treatments for Alzheimer's disease can be for either prophylactic or therapeutic purpose. When provided prophylactically, the treatments for Alzheimer's disease are provided in advance of any clinical symptom of Alzheimer's disease. Prophylactic administration serves to prevent or ameliorate any subsequent disease process. When provided therapeutically, the compounds are provided at (or shortly after) the onset of a symptom of disease.

For prophylactic and therapeutic purposes, the treatments for Alzheimer's disease can be administered to the subject in a single bolus delivery, via continuous delivery (for example, continuous transdermal, mucosal or intravenous delivery) over an extended time period, or in a repeated administration protocol (for example, by an hourly, daily or weekly, repeated administration protocol). The therapeutically effective dosage of the treatments for Alzheimer's disease can be provided as repeated doses within a prolonged prophylaxis or treatment regimen that will yield clinically significant results to alleviate one or more symptoms or detectable conditions associated with Alzheimer's disease.

Determination of effective dosages is typically based on animal model studies followed up by human clinical trials and is guided by administration protocols that significantly reduce the occurrence or severity of targeted disease symptoms or conditions in the subject. Suitable models in this regard include, for example, murine, rat, porcine, feline, non-human primate, and other accepted animal model subjects known in the art. Alternatively, effective dosages can be determined using in vitro models (for example, immunologic and histopathologic assays). Using such models, only ordinary calculations and adjustments are required to determine an appropriate concentration and dose to administer a therapeutically effective amount of the treatments for Alzheimer's disease (for example, amounts that are effective to alleviate one or more symptoms of Alzheimer's disease).

The actual dosages of treatments for Alzheimer's disease will vary according to factors such as the disease indication and particular status of the subject (for example, the subject's age, size, fitness, extent of symptoms, susceptibility factors, and the like), time and route of administration, other drugs or treatments being administered concurrently, as well as the specific pharmacology of treatments for Alzheimer's disease for eliciting the desired activity or biological response in the subject. Dosage regimens can be adjusted to provide an optimum prophylactic or therapeutic response.

A therapeutically effective amount is also one in which any toxic or detrimental side effects of the compound and/or other biologically active agent is outweighed in clinical terms by therapeutically beneficial effects. A non-limiting range for a therapeutically effective amount of treatments for Alzheimer's disease within the methods and formulations of the disclosure is about 0.0001 μg/kg body weight to about 10 mg/kg body weight per dose, such as about 0.0001 μg/kg body weight to about 0.001 μg/kg body weight per dose, about 0.001 μg/kg body weight to about 0.01 μg/kg body weight per dose, about 0.01 μg/kg body weight to about 0.1 μg/kg body weight per dose, about 0.1 μg/kg body weight to about 10 μg/kg body weight per dose, about 1 μg/kg body weight to about 100 μg/kg body weight per dose, about 100 μg/kg body weight to about 500 μg/kg body weight per dose, about 500 μg/kg body weight per dose to about 1000 μg/kg body weight per dose, or about 1.0 mg/kg body weight to about 10 mg/kg body weight per dose.

Dosage can be varied by the attending clinician to maintain a desired concentration. Higher or lower concentrations can be selected based on the mode of delivery, for example, trans-epidermal, rectal, oral, pulmonary, intranasal delivery, intravenous or subcutaneous delivery.

B. Detection of CCR6 Protein Molecules

In some embodiments of the disclosed methods, determining the amount of CCR6 expressed in a biological sample includes determining the amount of CCR6 protein, such as a CCR6 protein with an amino acid sequence at least 80% identical, such as at least 85% identical, at least 90% identical, at least 95% identical, as at least 98% identical, or even 100% identical to SEQ ID NO. 2 or 4 or a fragment thereof, in the biological sample. Exemplary amino acid sequences of murine and human CCR6 are given as SEQ ID NOs: 2 and 4, respectively:

MSGESMNFSDVFDSSEDYFVSVNTSYYSVDSEMLLCSLQEVRQFSRL
FVPIAYSLICVFGLLGNILVVITFAFYKKARSMTDVYLLNMAIADIL
FVLTLPFWAVSHATGAWVFSNATCKLLKGIYAINFNCGMLLLTCISM
DRYIAIVQATKSFRLRSRTLPRSKIICLVVWGLSVIISSSTFVFNQK
YNTQGSDVCEPKYQTVSEPIRWKLLMLGLELLFGFFIPLMFMIFCYT
FIVKTLVQAQNSKRHKAIRVIIAVVLVFLACQIPHNMVLLVTAANLG
KMNRSCQSEKLIGYTKTVTEVLAFLHCCLNPVLYAFIGQKFRNYFLK
ILKDLWCVRRKYKSSGFSCAGRYSENISRQTSETADNDNASSFTM;
(SEQ ID NO: 4; GENBANK ® ACCESSION NO. AAH37960
as available Jun. 5, 2010, which is hereby
incorporated by reference in its entirety);

MNSTESYFGTDDYDNTEYYSIPPDHGPCSLEEVRNFTKVFVPIAYSL
ICVFGLLGNIMVVMTFAFYKKARSMTDVYLLNMAITDILFVLTLPFW
AVTHATNTWVFSDALCKLMKGTYAVNFNCGMLLLACISMDRYIAIVQ
ATKSFRVRSRTLTHSKVICVAVWFISIIISSPTFIFNKKYELQDRDV
CEPRYRSVSEPITWKLLGMGLELFFGFFTPLLFMVFCYLFIIKTLVQ
AQNSKRHRAIRVVIAVVLVFLACQIPHNMVLLVTAVNTGKVGRSCST
EKVLAYTRNVAEVLAFLHCCLNPVLYAFIGQKFRNYFMKIMKDVWCM
RRKNKMPGFLCARVYSESYISRQTSETVENDNASSFTM;
(SEQ ID NO: 2; GENBANK ® ACCESSION NO. NP_033965
as available Jun. 5, 2010, which is hereby
incorporated by reference in its entirety);

CCR6 protein can be detected and the amount of CCR6 protein present in the biological sample can be quantified through novel epitopes recognized by polyclonal and/or monoclonal antibodies used in methods such as ELISA, immunoblot assays, flow cytometric assays, immunohistochemical assays, radioimmuno assays, Western blot assays, an immunofluorescent assays, chemiluminescent assays and other polypeptide detection strategies (Wong et al., Cancer Res., 46: 6029-6033, 1986; Luwor et al., Cancer Res., 61: 5355-5361, 2001; Mishima et al., Cancer Res., 61: 5349-5354, 2001; Ijaz et al., J. Med. Virol., 63: 210-216, 2001). Generally these methods utilize antibodies, such as monoclonal or polyclonal antibodies.

Generally, immunoassays for CCR6 typically include incubating a biological sample in the presence of antibody, and detecting the bound antibody by any of a number of techniques well known in the art. The biological sample can be peripheral blood including whole blood or any fraction thereof, including isolated peripheral blood mononuclear cells, or lymphoid tissue. The biological sample can also be isolated B cells, such as CD19+ B cells. The biological sample can be brought in contact with and immobilized onto a solid phase support or carrier such as or other solid support which is capable of immobilizing cells, cell particles or soluble proteins. The support may then be washed with suitable buffers followed by treatment with the antibody that binds CCR6. The solid phase support can then be washed with the buffer a second time to remove unbound antibody. If the antibody is directly labeled, the amount of bound label on solid support can then be detected by conventional means. If the antibody is unlabeled, a labeled second antibody, which detects that antibody that specifically binds CCR6 can be used.

A solid phase support may be any support capable of binding an antigen or an antibody. Well-known supports or carriers include glass, silocone dioxide or other silanes, polyvinyl, polystyrene, polypropylene, polyethylene, dextran, nylon, amylases, natural and modified celluloses, polyacrylamides, gabbros, hydrogels, gold, platinum, microbeads, micelles and other lipid formations, and magnetite. The nature of the carrier can be either soluble to some extent or insoluble for the purposes of the present disclosure. The support material may have virtually any possible structural configuration so long as the coupled molecule is capable of binding to an antigen or antibody. Thus, the support configuration may be spherical, as in a bead, or cylindrical, as in the inside surface of a test tube, or the external surface of a rod. Alternatively, the surface may be flat such as a sheet or test strip.

In one embodiment, proteins are isolated from a biological sample, such as a peripheral blood sample. In other embodiments, proteins are isolated from a lymphoid tissue sample, such as spleen tissue. In one embodiment, an enzyme linked immunosorbent assay (ELISA) is utilized to detect the protein (Voller, “The Enzyme Linked Immunosorbent Assay (ELISA),” Diagnostic Horizons 2:1-7, 1978, Microbiological Associates Quarterly Publication, Walkersville, Md.; Voller et al., J. Clin. Pathol. 31:507-520, 1978; Butler, Meth. Enzymol. 73:482-523, 1981; Maggio, (ed.) Enzyme Immunoassay, CRC Press, Boca Raton, Fla., 1980; Ishikawa, et al., (eds.) Enzyme Immunoassay, Kgaku Shoin, Tokyo, 1981). In this method, an enzyme which is bound to the antibody will react with an appropriate substrate, preferably a chromogenic substrate, in such a manner as to produce a chemical moiety which can be detected, for example, by spectrophotometric, fluorimetric or by visual means. Enzymes which can be used to detectably label the antibody include, but are not limited to, malate dehydrogenase, staphylococcal nuclease, delta-5-steroid isomerase, yeast alcohol dehydrogenase, alpha-glycerophosphate, dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase. The detection can be accomplished by colorimetric methods which employ a chromogenic substrate for the enzyme. Detection can also be accomplished by visual comparison of the extent of enzymatic reaction of a substrate in comparison with similarly prepared standards.

However, detection can also be accomplished using any of a variety of other immunoassays. For example, by radioactively labeling the antibodies or antibody fragments, it is possible to detect fingerprint gene wild-type or mutant peptides through the use of a radioimmunoassay (RIA) (see, for example, Weintraub, B., Principles of Radioimmunoassays, Seventh Training Course on Radioligand Assay Techniques, The Endocrine Society, March, 1986, which is incorporated by reference herein). In another example, a sensitive and specific tandem immunoradiometric assay may be used (see Shen and Tai, J. Biol. Chem., 261:25, 11585-11591, 1986). The radioactive isotope can be detected by such means as the use of a gamma counter or a scintillation counter or by autoradiography.

It is also possible to label the antibody with a fluorescent compound. When the fluorescently labeled antibody is exposed to light of the proper wavelength, its presence can then be detected due to fluorescence. Among the most commonly used fluorescent labeling compounds are fluorescein isothiocyanate, rhodamine, phycoerythrin, phycocyanin, allophycocyanin, o-phthaldehyde and fluorescamine. The antibody can also be detectably labeled using fluorescence emitting metals such as ¹⁵²Eu, or others of the lanthanide series. These metals can be attached to the antibody using such metal chelating groups as diethylenetriaminepentacetic acid (DTPA) or ethylenediaminetetraacetic acid (EDTA). The antibody also can be detectably labeled by coupling it to a chemiluminescent compound. The presence of the chemiluminescent-tagged antibody is then determined by detecting the presence of luminescence that arises during the course of a chemical reaction. Examples of particularly useful chemiluminescent labeling compounds are luminol, isoluminol, theromatic acridinium ester, imidazole, acridinium salt and oxalate ester. Likewise, a bioluminescent compound can be used to label the antibody of the present invention. Bioluminescence is a type of chemiluminescence found in biological systems in which a catalytic protein increases the efficiency of the chemiluminescent reaction. The presence of a bioluminescent protein is determined by detecting the presence of luminescence. Important bioluminescent compounds for purposes of labeling are luciferin, luciferase and aequorin.

In some embodiments, the amount of CCR6 protein present in the biological sample and thus the amount of CCR6 expressed is detected using a CCR6 protein specific binding agent, such as an antibody or ligand for CCR6, such as CCL20, which can be detectably labeled. In some embodiments, the specific binding agent is an antibody, such as a polyclonal or monoclonal antibody, that specifically binds CCR6 protein. Thus in certain embodiments, determining the amount of CCR6 expressed in a biological sample includes contacting a biological sample from the subject with a CCR6 protein specific binding agent (such as an antibody that specifically binds CCR6 protein), detecting whether the binding agent is bound by the sample, and thereby measuring the amount of CCR6 protein present in the sample. In certain embodiments, the CCR6 protein specific binding agent is an antibody or an antibody fragment that specifically binds CCR6 protein. In one embodiment, the specific binding agent is a monoclonal or polyclonal antibody that specifically binds the CCR6 protein.

An antibody that specifically binds a CCR6 protein typically binds with an affinity constant of at least 10⁷ M⁻¹, such as at least 10⁸ M⁻¹ at least 5×10⁸ M⁻¹ or at least 10⁹ M⁻¹. All antibodies that specifically bind CCR6 protein now known or yet to be developed are of use in the methods disclosed herein.

The preparation of polyclonal antibodies is well known to those skilled in the art. See, for example, Green et al., “Production of Polyclonal Antisera,” in: Immunochemical Protocols pages 1-5, Manson, ed., Humana Press 1992; Coligan et al., “Production of Polyclonal Antisera in Rabbits, Rats, Mice and Hamsters,” in: Current Protocols in Immunology, section 2.4.1, 1992.

The preparation of monoclonal antibodies likewise is conventional. See, for example, Kohler & Milstein, Nature 256:495, 1975; Coligan et al., sections 2.5.1-2.6.7; and Harlow et al., in: Antibodies: a Laboratory Manual, page 726, Cold Spring Harbor Pub., 1988. Briefly, monoclonal antibodies can be obtained by injecting mice with a composition including an antigen or a cell of interest, verifying the presence of antibody production by removing a serum sample, removing the spleen to obtain B lymphocytes, fusing the B lymphocytes with myeloma cells to produce hybridomas, cloning the hybridomas, selecting positive clones that produce antibodies to the antigen, and isolating the antibodies from the hybridoma cultures. Monoclonal antibodies can be isolated and purified from hybridoma cultures by a variety of well-established techniques. Such isolation techniques include affinity chromatography with Protein-A Sepharose, size-exclusion chromatography, and ion-exchange chromatography. See, e.g., Coligan et al., sections 2.7.1-2.7.12 and sections 2.9.1-2.9.3; Barnes et al., “Purification of Immunoglobulin G (IgG),” in: Methods in Molecular Biology, Vol. 10, pages 79-104, Humana Press, 1992.

Methods of in vitro and in vivo multiplication of monoclonal antibodies are well known to those skilled in the art. Multiplication in vitro may be carried out in suitable culture media such as Dulbecco's Modified Eagle Medium or RPMI 1640 medium, optionally supplemented by a mammalian serum such as fetal calf serum or trace elements and growth-sustaining supplements such as normal mouse peritoneal exudate cells, spleen cells, thymocytes or bone marrow macrophages. Production in vitro provides relatively pure antibody preparations and allows scale-up to yield large amounts of the desired antibodies. Large-scale hybridoma cultivation can be carried out by homogenous suspension culture in an airlift reactor, in a continuous stirrer reactor, or in immobilized or entrapped cell culture. Multiplication in vivo may be carried out by injecting cell clones into mammals histocompatible with the parent cells, e.g., syngeneic mice, to cause growth of antibody-producing tumors. Optionally, the animals are primed with a hydrocarbon, especially oils such as pristane (tetramethylpentadecane) prior to injection. After one to three weeks, the desired monoclonal antibody is recovered from the body fluid of the animal.

Humanized monoclonal antibodies are produced by transferring mouse complementarity determining regions from heavy and light variable chains of the mouse immunoglobulin into a human variable domain, and then substituting human residues in the framework regions of the murine counterparts. The use of antibody components derived from humanized monoclonal antibodies obviates potential problems associated with the immunogenicity of murine constant regions. General techniques for cloning murine immunoglobulin variable domains are described, for example, by Orlandi et al., Proc. Nat'l Acad. Sci. U.S.A. 86:3833, 1989. Techniques for producing humanized monoclonal antibodies are described, for example, by Jones et al., Nature 321:522, 1986; Riechmann et al., Nature 332:323, 1988; Verhoeyen et al., Science 239:1534, 1988; Carter et al., Proc. Nat'l Acad. Sci. U.S.A. 89:4285, 1992; Sandhu, Crit. Rev. Biotech. 12:437, 1992; and Singer et al., J. Immunol. 150:2844, 1993.

Antibodies include intact molecules as well as functional fragments thereof, such as Fab, F(ab′)₂, and Fv which are capable of binding the epitopic determinant. These antibody fragments retain some ability to selectively bind with their antigen. Methods of making these fragments are known in the art. (See for example, Harlow and Lane, Antibodies: A Laboratory Manual, Cold Spring Harbor Laboratory, New York, 1988).

Binding affinity for a target antigen is typically measured or determined by standard antibody-antigen assays, such as competitive assays, saturation assays, or immunoassays such as ELISA or RIA. Such assays can be used to determine the dissociation constant of the antibody. The phrase “dissociation constant” refers to the affinity of an antibody for an antigen. Specificity of binding between an antibody and an antigen exists if the dissociation constant (K_{D}=1/K, where K is the affinity constant) of the antibody is, for example <1 μM, <100 nM, or <0.1 nM. Antibody molecules will typically have a K_{D} in the lower ranges. K_{D}=[Ab−Ag]/[Ab][Ag] where [Ab] is the concentration at equilibrium of the antibody, [Ag] is the concentration at equilibrium of the antigen and [Ab−Ag] is the concentration at equilibrium of the antibody-antigen complex. Typically, the binding interactions between antigen and antibody include reversible noncovalent associations such as electrostatic attraction, Van der Waals forces and hydrogen bonds.

A monoclonal antibody with binding specificity for CCR6 is available from R&D Systems Clone #53103 Catalog Number MAB195, (technical data sheet available at http://www.rndsystems.com/pdf/mab195.pdf, last checked 3 Jun. 3, 2011, hereby incorporated by reference in its entirety.) Another monoclonal antibody with binding specificity for CCR6 protein is Becton Dickinson clone #11A9, Material Number 559560, (technical data sheet available at http://www.bdbiosciences.com/external_files/pm/doc/tds/brm/live/web enabled/23531D_{—}559560.pdf, last checked 3 Jun. 2011, hereby incorporated by reference in its entirety.) Yet another monoclonal antibody with binding specificity to CCR6 is eBioscience clone R6H1, Catalog Number 14-1969, (technical data sheet available at http://www.ebioscience.com/media/pdf/tds/14/14-1969.pdf last checked 3 Jun. 2011 and Carramolino et al, J. Leukoc. Biol. 66, 837-844 (1999), both of which are hereby incorporated by reference in its entirety). One skilled in the art will appreciate that there are other commercial sources for antibodies to CCR6 protein, in any of a number of forms including monoclonal antibodies, polyclonal antibodies, and any antibody fragment and/or conjugate thereof.

The antibodies used in the methods disclosed herein can be labeled. The enzymes that can be conjugated to the antibodies include, but are not limited to, alkaline phosphatase, peroxidase, urease and B-galactosidase. The fluorochromes that can be conjugated to the antibodies include, but are not limited to, fluorescein isothiocyanate, tetramethylrhodamine isothiocyanate, phycoerythrin, allophycocyanins and Texas Red. For additional fluorochromes that can be conjugated to antibodies see Haugland, R. P., Molecular Probes Handbook of Fluorescent Probes and Research Chemicals (1992-1994). The metal compounds that can be conjugated to the antibodies include, but are not limited to, ferritin, colloidal gold, and particularly colloidal superparamagnetic beads. The haptens that can be conjugated to the antibodies include, but are not limited to, biotin, digoxigenin, oxazalone, and nitrophenol. The radioactive compounds that can be conjugated or incorporated into the antibodies are known to the art, and include but are not limited to technetium 99m (⁹⁹Tc), ¹²⁵I and amino acids including any radionucleotides, including but not limited to, ¹⁴C, ³H and ³⁵S.

Any method known to those of skill in the art can be used to detect and quantify CCR6 protein. Thus, in additional embodiments, a spectrometric method is utilized. Spectrometric methods include mass spectrometry, nuclear magnetic resonance spectrometry, and combinations thereof. In one example, mass spectrometry is used to detect the presence of CCR6 protein in a biological sample, such as a blood sample, a serum sample, or a plasma sample (see for example, Stemmann, et al., Cell 107 715-726, 2001; Zhukov et al., “From Isolation to Identification: Using Surface Plasmon Resonance-Mass Spectrometry in Proteomics, PharmaGenomics, March/April 2002, available on the PharmaGenomics website on the internet).

CCR6 protein also can be detected by mass spectrometry assays for example coupled to immunaffinity assays, the use of matrix-assisted laser desorption/ionization time-of-flight (MALDI-TOF) mass mapping and liquid chromatography/quadrupole time-of-flight electrospray ionization tandem mass spectrometry (LC/Q-TOF-ESI-MS/MS) sequence tag of proteins separated by two-dimensional polyacrylamide gel electrophoresis (2D-PAGE) (Kiernan et al., Anal. Biochem. 301, 49-56 (2002); Poutanen et al., Mass Spectrom. 15, 1685-1692 (2001).

The presence of a CCR6 protein can be detected with multiple specific binding agents, such as one, two, three, or more specific binding agents. Thus, the methods can utilize more than one antibody. In some embodiments, one of the antibodies is attached to a solid support, such as a multiwell plate (such as, a microtiter plate), bead, membrane or the like. In practice, microtiter plates may conveniently be utilized as the solid phase. The surfaces may be prepared in advance, stored, and shipped to another location(s). However, antibody reactions also can be conducted in a liquid phase.

C. Detection of CCR6 Nucleic Acid Molecules

In some embodiments of the disclosed methods, determining the amount of CCR6 expressed in a biological sample includes determining the amount of CCR6 nucleic acid, such as CCR6 mRNA, in the biological sample. For example a CCR6 nucleic acid with an nucleic acid sequence at least 80% identical, such as at least 85% identical, at least 90% identical, at least 95% identical, as at least 98% identical, or even 100% identical to SEQ ID NO. 1 or 3 or a fragment thereof, in the biological sample. Exemplary nucleotide sequences of murine and human CCR6 are given as SEQ ID NOs: 1 and 3, respectively: GENBANK® ACCESSION NO. BC037960 as available Jun. 5, 2010, which is hereby incorporated by reference in its entirety).

Methods of determining the amount of nucleic acids, such as mRNA encoding CCR6 based on hybridization analysis and/or sequencing are known in the art. Methods known in the art for the quantification of mRNA expression in a sample include northern blotting and in situ hybridization (Parker & Barnes, Methods in Molecular Biology 106 247-283 (1999); RNAse protection assays (Hod, Biotechniques 13, 852-854 (1992)); and PCR-based methods, such as reverse transcription polymerase chain reaction (RT-PCR) (Weis et al., Trends in Genetics 8, 263-264 (1992)). Representative methods for sequencing-based gene expression analysis include Serial Analysis of Gene Expression (SAGE), and gene expression analysis by massively parallel signature sequencing (MPSS). (See Mardis E R, Annu. Rev. Genomics Hum Genet 9, 387-402 (2008)). In some embodiments, determining the amount of CCR6 expressed in a biological sample includes determining the amount of CCR6 mRNA in the biological sample.

Methods for quantitating mRNA are well known in the art. In one example, the method utilizes reverse transcriptase polymerase chain reaction (RT-PCR). Generally, the first step in gene expression profiling by RT-PCR is the reverse transcription of the RNA template into cDNA, followed by its exponential amplification in a PCR reaction. The two most commonly used reverse transcriptases are avian myeloblastosis virus reverse transcriptase (AMV-RT) and Moloney murine leukemia virus reverse transcriptase (MMLV-RT). The reverse transcription step is typically primed using specific primers, random hexamers, or oligo-dT primers, depending on the circumstances and the goal of expression profiling. For example, extracted RNA can be reverse-transcribed using a GENEAMP® RNA PCR kit (Perkin Elmer, Calif., USA), following the manufacturer's instructions. The derived cDNA can then be used as a template in the subsequent PCR reaction.

Although the PCR step can use a variety of thermostable DNA-dependent DNA polymerases, it typically employs the Taq DNA polymerase, which has a 5′-3′ nuclease activity but lacks a 3′-5′ proofreading endonuclease activity. Thus, TAQMAN® PCR typically utilizes the 5′-nuclease activity of Taq or Tth polymerase to hydrolyze a hybridization probe bound to its target amplicon, but any enzyme with equivalent 5′ nuclease activity can be used. Two oligonucleotide primers are used to generate an amplicon typical of a PCR reaction. A third oligonucleotide, or probe, is designed to detect nucleotide sequence located between the two PCR primers. The probe is non-extendible by Taq DNA polymerase enzyme, and is labeled with a reporter fluorescent dye and a quencher fluorescent dye. Any laser-induced emission from the reporter dye is quenched by the quenching dye when the two dyes are located close together as they are on the probe. During the amplification reaction, the Taq DNA polymerase enzyme cleaves the probe in a template-dependent manner. The resultant probe fragments disassociate in solution, and signal from the released reporter dye is free from the quenching effect of the second fluorophore. One molecule of reporter dye is liberated for each new molecule synthesized, and detection of the unquenched reporter dye provides the basis for quantitative interpretation of the data. Examples of fluorescent labels that may be used in quantitative PCR include but need not be limited to: HEX, TET, 6-FAM, JOE, Cy3, Cy5, ROX TAMRA, and Texas Red. Examples of quenchers that may be used in quantitative PCR include, but need not be limited to TAMRA (which may be used as a quencher with HEX, TET, or 6-FAM), BHQ1, BHQ2, or DABCYL

TAQMAN® RT-PCR can be performed using commercially available equipment, such as, for example, ABI PRISM 7700® Sequence Detection System™ (Perkin-Elmer-Applied Biosystems, Foster City, Calif., USA), or Lightcycler (Roche Molecular Biochemicals, Mannheim, Germany). In one embodiment, the 5′ nuclease procedure is run on a real-time quantitative PCR device such as the ABI PRISM 7700® Sequence Detection System. The system includes of thermocycler, laser, charge-coupled device (CCD), camera and computer. The system amplifies samples in a 96-well format on a thermocycler. During amplification, laser-induced fluorescent signal is collected in real-time through fiber optics cables for all 96 wells, and detected at the CCD. The system includes software for running the instrument and for analyzing the data.

In some examples, 5′-nuclease assay data are initially expressed as Ct, or the threshold cycle. As discussed above, fluorescence values are recorded during every cycle and represent the amount of product amplified to that point in the amplification reaction. The point when the fluorescent signal is first recorded as statistically significant is the threshold cycle (Ct).

To minimize errors and the effect of sample-to-sample variation, RT-PCR can be performed using an internal standard. The ideal internal standard is expressed at a constant level among different tissues, and is unaffected by the experimental treatment. RNAs most frequently used to normalize patterns of gene expression are the mRNA products of housekeeping genes.

Generally, with regard to nucleic acids, any method can be utilized provided it can detect the expression of target gene mRNA (CCR6) as compared to a control. One of skill in the art can readily identify an appropriate control, such as a sample from a subject known not to have a disorder (a negative control), a sample from a subject known to have a disorder (a positive control), or a known amount of nucleic acid encoding CCR6 (a standard or a normal level found in a healthy subject). Statistically normal levels can be determined for example, from a subject with known not be have Alzheimer's disease.

While the invention encompasses any primer/probe set appropriate for the determination of CCR6 expression level by TaqMan® analysis, some examples are as follows: Hs01890706_s1 (Amplicon length 145) Hs99999079_m1 (Amplicon length 73), Hs01853366_s1 (Amplicon length 142), Hs00171121_m1 (Amplicon length 63). All of the listed Primer/Probe sets may be obtained from Applied Biosystems. Examples of additional primer/probe sets that may be used in TaqMan® analysis include, but need not be limited to the following:

| PRIMER/PROBE SET NO: 1 | |
|---|---|
| SEQ ID NO: 5 | |
| Forward: | TTGAAGGACCTGTGGTGTGT |
| SEQ ID NO: 6 | |
| Reverse: | TTGTCGTTATCTGCGGTCTC |
| SEQ ID NO: 7 | |
| Probe: | CCTCCCGGCACAGGAGAAGC |

The probe in PRIMER/PROBE SET NO: 1 may be modified with 5′-FAM and 3′-TAMRA
PRIMER/PROBE SET NO: 1 yields a 113 base pair amplicon.

| PRIMER/PROBE SET NO: 2 | |
|---|---|
| SEQ ID NO: 8 | |
| Forward: | GGTGAGCTGGAGTCATCAGA |
| SEQ ID NO: 9 | |
| Reverse: | GTGACTCTCAGGCAGTGCTC |
| SEQ ID NO: 10 | |
| Probe: | CCTTCAGCCTCACTCCGGGC |

The probe in PRIMER/PROBE SET NO: 2 may be modified with 5′-FAM and 3′-TAMRA
PRIMER/PROBE SET NO: 2 yields a 77 base pair amplicon.

| PRIMER/PROBE SET NO: 3 | |
|---|---|
| SEQ ID NO: 11 | |
| Forward: | GACCAGTGAGACCGCAGATA |
| SEQ ID NO: 12 | |
| Reverse: | TCACACATGCCTTAGGGAGA |
| SEQ ID NO: 13 | |
| Probe: | CGACAATGCGTCGTCCTTCACTATG |

The probe in PRIMER/PROBE SET NO: 3 may be modified with 5′-FAM and 3′-TAMRA
PRIMER/PROBE SET NO: 3 yields a 81 base pair amplicon.

Additionally, quantitative PCR may be performed upon a cDNA resulting from the reverse transcription of a sample from a subject without the use of a labeled oligonucleotide probe that binds to a sequence between the primers. In some of these techniques, PCR amplification is tracked by the binding of a fluorescent dye such as SYBR green to the double stranded PCR product during the amplification reaction. SYBR green binds to double stranded DNA, but not to single stranded DNA. In addition, SYBR green fluoresces strongly at a wavelength of 497 nm when it is bound to double stranded DNA, but does not fluoresce when it is not bound to double stranded DNA. As a result, the intensity of fluorescence at 497 nm may be correlated with the amount of amplification product present at any time during the reaction. The rate of amplification may in turn be correlated with the amount of template sequence present in the initial sample. Generally, Ct values are calculated similarly to those calculated using the TaqMan® system. Because the probe is absent, amplification of the proper sequence may be checked by any of a number of techniques. One such technique involves running the amplification products on an agarose or other gel appropriate for resolving nucleic acid fragments and comparing the amplification products from the quantitative real time PCR reaction with control DNA fragments of known size.

While the invention encompasses any primer set that is that is appropriate for the determination of CCR6 expression level by quantitative PCR without the use of labeled probes, some examples are as follows:

| Primer Set NO: 1 | |
|---|---|
| SEQ ID NO: 14 | |
| Forward Primer: | GAGGTCAGGCAGTTCTCCAG |
| SEQ ID NO: 15 | |
| Reverse Primer: | GCTGCCTTGGGTGTTGTATT |
| Amplicon Size = 465 | |
| Primer Set NO: 2 | |
| SEQ ID NO: 16 | |
| Forward Primer: | CAGGAGGTCAGGCAGTTCTC |
| SEQ ID NO: 15 | |
| Reverse Primer: | GCTGCCTTGGTGTTGTATT |
| Amplicon Size = 468 | |
| Primer Set NO: 3 | |
| SEQ ID NO: 17 | |
| Forward Primer: | GGCTGCAATTTGGGTAAAA |
| SEQ ID NO: 18 | |
| Reverse Primer: | CACAGGAGAAGCCTGAGGAC |
| Amplicon Size = 215 | |
| Primer Set NO: 4 | |
| SEQ ID NO: 19 | |
| Forward Primer: | GAGGTCAGGCAGTTCTCCAG |
| SEQ ID NO: 20 | |
| Reverse Primer: | GGATGGCTTTGTGCCTTTTA |
| Amplicon Size = 643 | |
| Primer Set NO: 5 | |
| SEQ ID NO: 16 | |
| Forward Primer: | CAGGAGGTCAGGCAGTTCTC |
| SEQ ID NO: 20 | |
| Reverse Primer: | GGATGGCTTTGTGCCTTTTA |

Amplicon Size = 646

An expression level of CCR6 in a sample may be quantified in comparison to an internal standard such as a housekeeping gene. When housekeeping gene expression is determined in the same sample as CCR6, CCR6 expression may be normalized to the expression of the housekeeping gene. So expression of the housekeeping gene serves as an internal normalization control that serves to reduce sample-to-sample variability with regard to CCR6 expression. A housekeeping gene may be any gene that is constitutively expressed in most or all tissues in an organism at a constant level of expression. See Eisenberg and Levanon, Trends in Genetics 19, 362-365 (2003), hereby incorporated by reference in its entirety.) A list of human housekeeping genes is available at http://www.compugen.co.il/supp_info/Housekeeping_genes.html, last checked 3 Jun., 2011, (list and the contents of all Genbank references identified by accession number are hereby incorporated by reference in their entirety.) One of skill in the art would know how to select one or more acceptable housekeeping genes to be used in any method of assessing CCR6 expression.

One such housekeeping gene that may be used in determining the expression of CCR6 is human beta actin (SEQ ID NO. 21.) Human beta actin may be used as a housekeeping gene in determining the expression of CCR6 by TaqMan® PCR, or any appropriate method. While the invention encompasses any primer/probe set that may amplify beta-actin (or any other housekeeping gene), some examples are as follows: Hs01890706_s1 (Amplicon length 145), Hs99999079_m1 (Amplicon length 73), Hs01853366_s1 (Amplicon length 142), Hs00171121_m1 (Amplicon length 63). All of the above are available from Applied Biosystems. Additional examples of primer/probe sets that may be used to amplify beta-actin sequences in TaqMan® analysis include:

| PRIMER/PROBE SET NO: 4 | |
|---|---|
| SEQ ID NO: 22 | |
| Forward Primer: | TGGACTTCGAGCAAGAGATG |
| SEQ ID NO: 23 | |
| Reverse Primer: | GAAGGAAGGCTGGAAGAGTG |
| SEQ ID NO: 24 | |
| Probe: | CGGCTGCTTCCAGCTCCTCC |

The probe in PRIMER/PROBE SET NO: 4 may be modified with 5′-FAM and 3′-TAMRA
PRIMER/PROBE SET NO: 4 yields a 137 base pair amplicon.

| PRIMER/PROBE SET NO: 5 | |
|---|---|
| SEQ ID NO: 25 | |
| Forward Primer: | GCACCCAGCACAATGAAG |
| SEQ ID NO: 26 | |
| Reverse Primer: | CGATCCACACGGAGTACTTG |
| SEQ ID NO: 27 | |
| Probe: | CAAGATCATTGCTCCTCCTGAGCG |

The probe in PRIMER/PROBE SET NO: 5 may be modified with 5′-FAM and 3′-TAMRA
PRIMER/PROBE SET NO: 5 yields a 64 base pair amplicon.

| PRIMER/PROBE SET NO: 6 | |
|---|---|
| SEQ ID NO: 28 | |
| Forward Primer: | GGCATGGGTCAGAAGGATT |
| SEQ ID NO: 29 | |
| Reverse Primer: | AGAAGGTGTGGTGCCAGATT |
| SEQ ID NO: 30 | |
| Probe: | CATCGAGCACGGCATCGTCA |

The probe in PRIMER/PROBE SET NO: 6 may be modified with 5′-FAM and 3′-TAMRA
PRIMER/PROBE SET NO: 6 yields a 136 base pair amplicon.

The methods described herein may be performed, for example, by utilizing diagnostic kits comprising at least one specific nucleic acid probe, which may be conveniently used, such as in clinical settings, to diagnose subjects exhibiting cardiovascular disease symptoms or at risk for developing AD. Such kits may be provided in the form of a package, box, bag, or other container enclosing one or more components that may be used in determining the expression of CCR6. Such kits may also contain labeling reagents, enzymes including PCR amplification reagents such as Taq or Pfu; reverse transcriptase and additional buffers and solutions that facilitate the performance of the method.

A diagnostic kit may contain reagents, such as antibodies, that specifically bind proteins. Such kits will contain one or more specific antibodies, buffers, and other reagents configured to detect binding of the antibody to the specific epitope. One or more of the antibodies may be labeled with a fluorescent, enzymatic, magnetic, metallic, chemical, or other label that signifies and/or locates the presence of specifically bound antibody. The kit may also contain one or more secondary antibodies that specifically recognize epitopes on other antibodies. These secondary antibodies may also be labeled. The concept of a secondary antibody also encompasses non-antibody ligands that specifically bind an epitope or label of another antibody. For example, streptavidin or avidin may bind to biotin conjugated to another antibody. Such a kit may also contain enzymatic substrates that change color or some other property in the presence of an enzyme that is conjugated to one or more antibodies included in the kit.

Kits may be provided as a reagent bound to a substrate material. For example, the kit may comprise an antibody or other protein reagent bound to a polystyrene plate. Alternatively, the kit may comprise a nucleic acid such as an oligonucleotide, bound to a substrate, wherein a substrate may be any solid or semi solid material onto which a nucleic acid, such as an oligonucleotide may be affixed, attached or printed, either singly or in a microarray format.

Examples of substrate materials include but are not limited to polyvinyl, polysterene, polypropylene, polyester or any other plastic, glass, silicon dioxide or other silanes, hydrogels, gold, platinum, microbeads, micelles and other lipid formations, nitrocellulose, or nylon membranes. The substrate may take any form, including a spherical bead or flat surface.

A diagnostic kit may also contain an indication of a threshold level of expression of CCR6 that will signify that the subject has AD. An indication may be any communication of a threshold level of expression. The indication may further indicate that expression of CCR6 above the threshold level of expression will signify that the subject has AD. The indication of the threshold level may be provided in multiple stages such in a system that the subject has a high, medium or low risk of having AD. The indication may comprise any number of stages. The indication may indicate the threshold of expression numerically, as in an optical density of an ELISA assay, a protein concentration (such as ng/ml), a percentage of cells expressing CCR6, or in fold-expression relative to a positive control, negative control, or housekeeping gene. The indication may be a positive or negative control that intended to be matched to the sample by eye or through an instrument. The indication may be a size marker to be compared to the sample through gel electrophoresis.

The indication may be communicated through any tangible medium of expression. It may be printed the packaging material, a separate piece of paper, or any other substrate and provided with the kit, provided separately from the kit, posted on the Internet, written into a software package. The indication may comprise an image such as a FACS image, a photograph or a photomicrograph, or any copy or other reproduction of these, particularly when CCR6 expression is determined through the use of in situ hybridization, FACS analysis, or immunohistochemistry,

The diagnostic procedures can be performed “in situ” directly upon blood smears (fixed and/or frozen), or on tissue biopsies, such that no nucleic acid purification is necessary. DNA or RNA from a sample can be isolated using procedures which are well known to those in the art.

Nucleic acid reagents that are specific to the nucleic acid of interest, namely the nucleic acid encoding CCR6, can be readily generated given the sequences of these genes for use as probes and/or primers for such in situ procedures (see, for example, Nuovo, G. J., 1992, PCR in situ hybridization: protocols and applications, Raven Press, NY).

In one embodiment, a nucleic acid sample is utilized, such as the total mRNA isolated from a biological sample. The biological sample can be from any biological tissue or fluid from the subject of interest, such as a subject who is suspected of having cardiovascular disease. Such samples include, but are not limited to, blood, blood cells (such as white blood cells) or tissue biopsies including spleen tissue.

Nucleic acids (such as mRNA) can be isolated from the sample according to any of a number of methods well known to those of skill in the art. Methods of isolating total mRNA are well known to those of skill in the art. For example, methods of isolation and purification of nucleic acids are described in detail in Chapter 3 of Laboratory Techniques in Biochemistry and Molecular Biology: Hybridization With Nucleic Acid Probes, Part I. Theory and Nucleic Acid Preparation, P. Tijssen, ed. Elsevier, N.Y. (1993) and Chapter 3 of Laboratory Techniques in Biochemistry and Molecular Biology: Hybridization With Nucleic Acid Probes, Part I. Theory and Nucleic Acid Preparation, P. Tijssen, ed. Elsevier, N.Y. (1993). In one example, the total nucleic acid is isolated from a given sample using, for example, an acid guanidinium-phenol-chloroform extraction method, and polyA+ mRNA is isolated by oligo dT column chromatography or by using (dT)n magnetic beads (see, for example, Sambrook et al, Molecular Cloning: A Laboratory Manual (2nd ed.), Vols. 1-3, Cold Spring Harbor Laboratory, (1989), or Current Protocols in Molecular Biology, F. Ausubel et al., ed. Greene Publishing and Wiley-Interscience, N.Y. (1987)). In another example, oligo-dT magnetic beads may be used to purify mRNA (Dynal Biotech Inc., Brown Deer, Wis.). Nucleic acid may be isolated from blood either by lysing cells in whole blood prior to nucleic acid isolation or it may be isolated from a fraction of whole blood, such as PBMC.

The nucleic acid sample can be amplified prior to hybridization. If a quantitative result is desired, a method is utilized that maintains or controls for the relative frequencies of the amplified nucleic acids. Methods of “quantitative” amplification are well known to those of skill in the art. For example, quantitative PCR involves simultaneously co-amplifying a known quantity of a control sequence using the same primers. This provides an internal standard that can be used to calibrate the PCR reaction. The array can then include probes specific to the internal standard for quantification of the amplified nucleic acid.

Primers and probes used in quantitative PCR may be oligonucleotides. Oligonucleotide synthesis is the chemical synthesis of oligonucleotides with a defined chemical structure and/or nucleic acid sequence by any method now known in the art or yet to be disclosed. Oligonucleotide synthesis may be carried out by the addition of nucleotide residues to the 5′-terminus of a growing chain. Elements of oligonucleotide synthesis include: De-blocking (detritylation): A DMT group is removed with a solution of an acid, such as TCA or Dichloroacetic acid (DCA), in an inert solvent (dichloromethane or toluene) and washed out, resulting in a free 5′ hydroxyl group on the first base. Coupling: A nucleoside phosphoramidite (or a mixture of several phosphoramidites) is activated by an acidic azole catalyst, tetrazole, 2-ethylthiotetrazole, 2-bezylthiotetrazole, 4,5-dicyanoimidazole, or a number of similar compounds. This mixture is brought in contact with the starting solid support (first coupling) or oligonucleotide precursor (following couplings) whose 5′-hydroxy group reacts with the activated phosphoramidite moiety of the incoming nucleoside phosphoramidite to form a phosphite triester linkage. The phosphoramidite coupling may be carried out in anhydrous acetonitrile. Unbound reagents and by-products may be removed by washing.

A small percentage of the solid support-bound 5′-OH groups (0.1 to 1%) remain unreacted and should be permanently blocked from further chain elongation to prevent the formation of oligonucleotides with an internal base deletion commonly referred to as (n−1) shortmers. This is done by acetylation of the unreacted 5′-hydroxy groups using a mixture of acetic anhydride and 1-methylimidazole as a catalyst. Excess reagents are removed by washing.

The newly formed tricoordinated phosphite triester linkage is of limited stability under the conditions of oligonucleotide synthesis. The treatment of the support-bound material with iodine and water in the presence of a weak base (pyridine, lutidine, or collidine) oxidizes the phosphite triester into a tetracoordinated phosphate triester, a protected precursor of the naturally occurring phosphate diester internucleosidic linkage. This step can be substituted with a sulfurization step to obtain oligonucleotide phosphorothioates. In the latter case, the sulfurization step is carried out prior to capping. Upon the completion of the chain assembly, the product may be released from the solid phase to solution, deprotected, and collected. Products may be isolated by HPLC to obtain the desired oligonucleotides in high purity.

In one embodiment, the hybridized nucleic acids are detected by detecting one or more labels attached to the sample nucleic acids. The labels can be incorporated by any of a number of methods. In one example, the label is simultaneously incorporated during the amplification step in the preparation of the sample nucleic acids. Thus, for example, polymerase chain reaction (PCR) with labeled primers or labeled nucleotides will provide a labeled amplification product. In one embodiment, transcription amplification, as described above, using a labeled nucleotide (such as fluorescein-labeled UTP and/or CTP) incorporates a label into the transcribed nucleic acids.

Alternatively, a label may be added directly to the original nucleic acid sample (such as mRNA, polyA mRNA, cDNA, etc.) or to the amplification product after the amplification is completed. Means of attaching labels to nucleic acids are well known to those of skill in the art and include, for example, nick translation or end-labeling (e.g. with a labeled RNA) by kinasing of the nucleic acid and subsequent attachment (ligation) of a nucleic acid linker joining the sample nucleic acid to a label (e.g., a fluorophore).

Detectable labels suitable for use include any composition detectable by spectroscopic, photochemical, biochemical, immunochemical, electrical, optical or chemical means. Useful labels include biotin for staining with labeled streptavidin conjugate, magnetic beads (for example DYNABEADS™), fluorescent dyes (for example, fluorescein, Texas red, rhodamine, green fluorescent protein, and the like), radiolabels (for example, ³H, ¹²⁵I, ³⁵S, ¹⁴C, or ³²P), enzymes (for example, horseradish peroxidase, alkaline phosphatase and others commonly used in an ELISA), and colorimetric labels such as colloidal gold or colored glass or plastic (for example, polystyrene, polypropylene, latex, etc.) beads. Patents teaching the use of such labels include U.S. Pat. No. 3,817,837; U.S. Pat. No. 3,850,752; U.S. Pat. No. 3,939,350; U.S. Pat. No. 3,996,345; U.S. Pat. No. 4,277,437; U.S. Pat. No. 4,275,149; and U.S. Pat. No. 4,366,241.

Methods of detecting such labels are also well known. Thus, for example, radiolabels may be detected using photographic film or scintillation counters, fluorescent markers may be detected using a photodetector to detect emitted light. Enzymatic labels are typically detected by providing the enzyme with a substrate and detecting the reaction product produced by the action of the enzyme on the substrate, and colorimetric labels are detected by simply visualizing the colored label.

The label may be added to the target (sample) nucleic acid(s) prior to, or after, the hybridization. So-called “direct labels” are detectable labels that are directly attached to or incorporated into the target (sample) nucleic acid prior to hybridization. In contrast, so-called “indirect labels” are joined to the hybrid duplex after hybridization. Often, the indirect label is attached to a binding moiety that has been attached to the target nucleic acid prior to the hybridization. Thus, for example, the target nucleic acid may be biotinylated before the hybridization. After hybridization, an avidin-conjugated fluorophore will bind the biotin bearing hybrid duplexes providing a label that is easily detected (see Laboratory Techniques in Biochemistry and Molecular Biology, Vol. 24: Hybridization With Nucleic Acid Probes, P. Tijssen, ed. Elsevier, N.Y., 1993).

Nucleic acid hybridization simply involves providing a denatured probe and target nucleic acid under conditions where the probe and its complementary target can form stable hybrid duplexes through complementary base pairing. The nucleic acids that do not form hybrid duplexes are then washed away leaving the hybridized nucleic acids to be detected, typically through detection of an attached detectable label. It is generally recognized that nucleic acids are denatured by increasing the temperature or decreasing the salt concentration of the buffer containing the nucleic acids. Under low stringency conditions (e.g., low temperature and/or high salt) hybrid duplexes (e.g., DNA:DNA, RNA:RNA, or RNA:DNA) will form even where the annealed sequences are not perfectly complementary. Thus, specificity of hybridization is reduced at lower stringency. Conversely, at higher stringency (e.g., higher temperature or lower salt) successful hybridization requires fewer mismatches. One of skill in the art will appreciate that hybridization conditions can be designed to provide different degrees of stringency.

In general, there is a tradeoff between hybridization specificity (stringency) and signal intensity. Thus, in one embodiment, the wash is performed at the highest stringency that produces consistent results and that provides a signal intensity greater than approximately 10% of the background intensity. Thus, the hybridized array may be washed at successively higher stringency solutions and read between each wash. Analysis of the data sets thus produced will reveal a wash stringency above which the hybridization pattern is not appreciably altered and which provides adequate signal for the particular oligonucleotide probes of interest. These steps have been standardized for commercially available array systems.

Methods for evaluating the hybridization results vary with the nature of the specific probe nucleic acids used as well as the controls provided. In one embodiment, simple quantification of the fluorescence intensity for each probe is determined. This is accomplished simply by measuring probe signal strength at each location (representing a different probe) on the array (for example, where the label is a fluorescent label, detection of the amount of florescence (intensity) produced by a fixed excitation illumination at each location on the array). Comparison of the absolute intensities of an array hybridized to nucleic acids from a “test” sample (such as from a subject treated with a therapeutic protocol) with intensities produced by a “control” sample (such as from the same subject prior to treatment with the therapeutic protocol) provides a measure of the relative expression of the nucleic acids that hybridize to each of the probes.

Changes in expression detected by these methods for instance can be different for different therapies, and may include increases or decreases in the level (amount) or functional activity of such nucleic acids, their expression or translation into protein, or in their localization or stability. An increase or a decrease can be, for example, about a 1-fold, 2-fold, 3-fold, 4-fold, 5-fold, change (increase or decrease) in the expression of a particular nucleic acid, such as a nucleic acid encoding CCR6.

Alterations, including increases or decreases in the expression of nucleic acid molecules can be detected using, for instance, in vitro nucleic acid amplification and/or nucleic acid hybridization. The results of such detection methods can be quantified, for instance by determining the amount of hybridization or the amount of amplification.

An alternative quantitative nucleic acid amplification procedure is described in U.S. Pat. No. 5,219,727, which is incorporated herein by reference. In this procedure, the amount of a target sequence in a sample is determined by simultaneously amplifying the target sequence and an internal standard nucleic acid segment. The amount of amplified DNA from each segment is determined and compared to a standard curve to determine the amount of the target nucleic acid segment that was present in the sample prior to amplification.

### III. Examples

### Example 1

### Materials and Methods

The following Materials and Methods pertain to Example 2 Example 3, and Example 4.

Mice:

The 3×Tg-AD mouse is a model system for human Alzheimer's disease. 3×Tg-AD mice overexpress mutant forms of APP, presenilin-1, and tau known to be important in AD pathology. The mice come down with symptoms similar to humans with AD (See Oddo S et al, Neuron 39, 409-421 (2003), hereby incorporated by reference in its entirety.) WT and 3×Tg-AD (12-15 and 5-6 month old) mice were generated from breeding pairs. Mice were maintained in a climate controlled environment with a 12-hr light/12-hr dark cycle, and fed AIN-93M Purified Rodent Diet (Dyets Inc, Bethlehem, Pa.). Diet and water were supplied ad libitum. All procedures were conducted in accordance with the NIH Guidelines for the Care and Use of Laboratory Animals and were approved by the institutional Animal Care and Use Committee of the Portland VA Medical Center. Table 1 shows the cohorts of mice used and the number of mice per cohort.

TABLE 1

| Genotype | Age | Gender | Number of mice |
|---|---|---|---|
| WT | 12 months | Female | 3 |
| 3xTg-AD | 12 months | Female | 3 |
| WT | 5-6 months | Female | 9 |
| 3xTg-AD | 5-6 months | Female | 9 |
| WT | 5-6 months | Male | 9 |
| 3xTg-AD | 5-6 months | Male | 9 |

Isolation of Mononuclear Cells from Spleen, Blood and Brain:

Spleen and brain were isolated from all mice. Single cell suspensions were prepared by passing the tissue through a 100 μm nylon mesh screen. Spleen mononuclear cells were washed with RPMI medium and red cells were lysed using 1× red cell lysis buffer (eBiosciences, San Diego, Calif.). The mononuclear cells were washed twice, counted and resuspended in a stimulation medium containing 10% fetal bovine serum (FBS). Central nervous system (CNS) mononuclear cells were isolated by Percoll gradient centrifugation as described in Bebo B F et al, J. Neurosci Res 45, 680-689 (1996), (hereby incorporated by reference in its entirety.) Whole cardiac blood was collected in EDTA. The red cells were lysed and the remaining mononuclear cells pelleted. These were washed, counted and resuspended in stimulation medium containing 10% FBS.

Cytokine Detection by Luminex® Bead Array:

Single-cell suspensions of mononuclear cells were cultured in the presence of plate bound anti-CD3 (5m) and anti-CD28 (1 μg) mAb for 24 hours. Culture supernatants were collected and assessed for cytokine levels using a Luminex Bio-Plex® cytokine assay kit (Bio-Rad, Richmond, Calif.) following the manufacturer's instructions. Expression of the following cytokines was determined: IL-2, IL-6, IL-10, IL-13, IL-17, IFN-γ, MCP-1 and TNF-α.

RNA Isolation and Reverse Transcription-Polymerase Chain Reaction:

Total RNA was isolated from spleen mononuclear cells and brain mononuclear cells using the RNeasy® mini kit protocol (Qiagen, Valencia, Calif., USA) and converted into cDNA using oligo-dT, random hexamers, and Superscript® RT II (Invitrogen, Grand Island, N.Y., USA). Reverse transcription-PCR was performed using TaqMan® PCR master mix (Applied Biosystems, Foster City, Calif., USA) and primers. Reactions were conducted on the ABI Prism 7000 Sequence Detection System® (Applied Biosystems) to detect mRNA quantified as relative units compared with the β-Actin housekeeping gene. Pre-designed Taqman® primer/probe sets that specifically amplify each of ICAM-1, VCAM-1, IL-10, IL-2, IL-6, IL-10, IL-17α, TNF-α, dysferlin, Foxp3, CCL20, CCR2, CCR3, CCR4, CCR5, CCR6, CCR7, and CCR8 were obtained from Applied Biosystems (Foster City, Calif.).

### Statistical Analyses:

Statistical differences between groups were determined by Student's t test. A p value ≦0.05 was considered significant.

### Example 2

### Distribution of Cell Subsets in Spleen, Blood, and Brain of 3×Tg-AD Mice

Although brain pathology and associated changes have been extensively studied in the 3×Tg-AD mouse model, few, if any studies explored the role of inflammatory processes in the periphery, including the spleen and blood. Additionally, few, if any studies have explored the development of inflammatory processes in 5-6 month old 3×Tg-AD mice. It is of particular importance to determine the expression of biomarkers in these mice due to the fact that 5-6 month old mice have yet to show symptoms of disease. Biomarkers that signify the presence of AD prior to the onset of symptoms would be invaluable tools for clinicians in the treatment of AD.

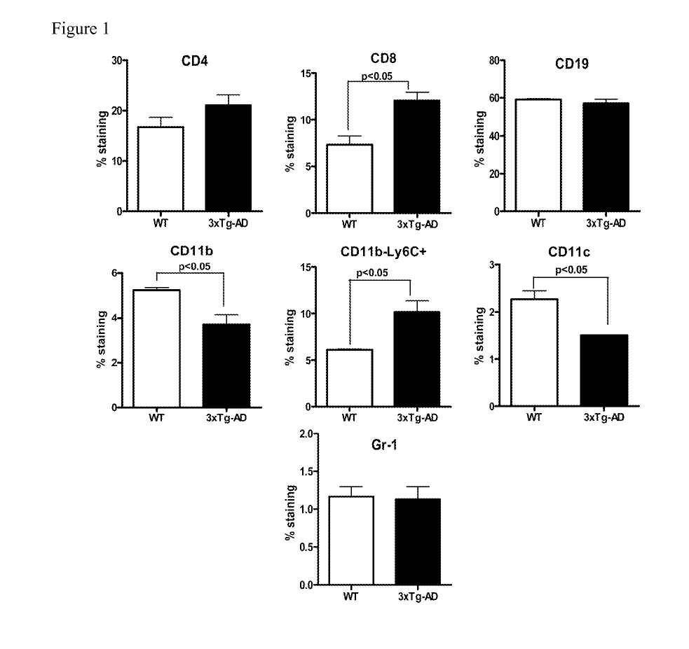

In order to determine if 3×Tg-AD mice show an altered inflammatory cell profile compared to their WT counterparts, splenocytes from 12-15 month-old female mice were stained for expression of the following markers to identify T and B cells (CD4+, CD8+ and CD19+), macrophages (CD11b+), dendritic cells (DC, CD11c+) and granulocytes (Gr-1+). The 3×Tg-AD mice showed a markedly higher percentage of CD8+ T cells (FIG. 1) in comparison to WT and a small but significant lower percentage of macrophages and DCs in spleen (FIG. 1). Data in FIG. 1 are presented as the mean±Standard deviation of 3 mice per group. Splenocytes from 3×Tg-AD mice comprised a higher percentage of Ly6C+ CD11b-cells (FIG. 1) relative to WT. Ly6C is a biomarker that signifies immunological memory. It is likely that this cell population is largely made up of CD8+ T cells. (See Walunas T et al, J Immunol 155, 1873-1883 (1995) and Lin S J et al, J Exp Med 204, 2321-2333 (2007), both of which are hereby incorporated by reference in their entireties.)

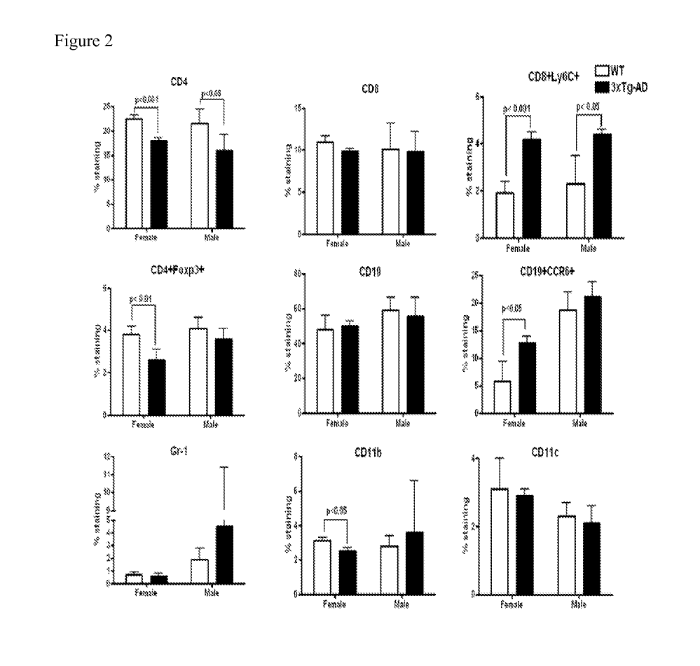

The distribution of inflammatory subtypes was also evaluated in spleen, blood and brain of younger (5-6 month old) male and female mice. Both male and female 3×Tg-AD mice had a significantly lower percentage of spleen CD4+ T cells than WT. Younger female 3×Tg-AD mice had a significantly lower percentage of spleen macrophages relative to WT. The lower percentage of macrophages was similar to that seen in 12 month old female 3×Tg-AD mice (FIG. 2). Both female and male 3×Tg-AD mice had a significantly higher percentage of Ly6C+ CD8+ T cells than WT, even though the total percentage of CD8+ T cells was the same as that observed in WT.

Splenocytes from the 5-6 month old female 3×Tg-AD mice had a higher percentage of CD19+ B cells expressing the chemokine receptor CCR6 relative to wild type. The 5-6 month old female 3×Tg-AD mice also showed a lower percentage of Foxp3+ Treg cells relative to WT (FIG. 2). In FIG. 2, the data are presented as the mean±the standard deviation of one of three replicated experiments each involving a total of 7-9 mice per group.

The 5-6 month 3×Tg-AD mice had a significantly lower percentage of CD4+ T cells in blood than WT. They also had a significantly higher percentage of Ly6C-expressing CD8+ cells in both females (p<0.001) and males (p<0.01). A higher percentage of blood B cells expressing CCR6 was seen in both male and female 3×Tg-AD mice relative to wild type, but the difference only rose to the defined level of significance in males.

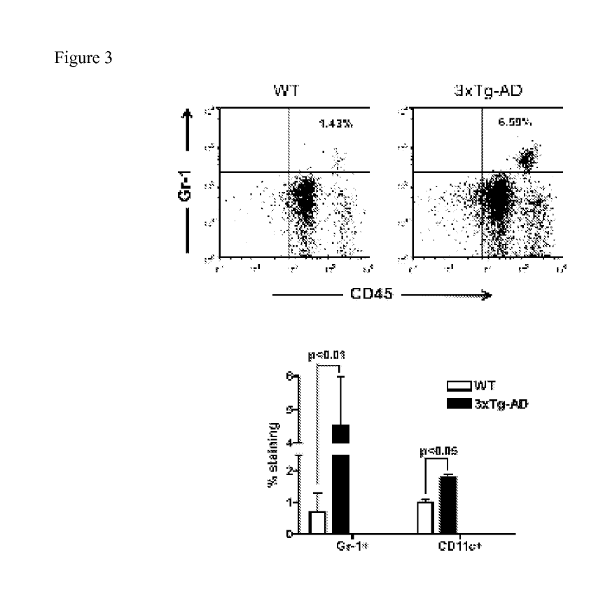

Brains of 5-6 month old 3×Tg-AD mice and age- and gender-matched WT mice were pooled and mononuclear cells isolated on a Percoll gradient. The cells were stained using labeled antibodies with specificity to T cells, B cells, macrophages, microglia, dendritic cells, and granulocytes. Results are shown in FIG. 3. The top panel is a plot of staining of CD45 on the x axis and Gr-1 on the Y-axis. The data presented in the graph in the bottom panel are the mean±the standard deviation of each of two experiments, each experiment consisting of pooled brain cells from WT and 3×Tg-AD mice. Pools of cells from WT mice included cells from three individual animals and pools from 3×Tg-AD included cells from four individual animals.

Immune cells isolated from the brains of 5-6 month old 3×Tg-AD mice showed a higher percentage of Gr1+ granulocytes relative to WT. Additionally, there was a higher percentage of CD11c+ DC isolated from the brains of female 3×Tg-AD relative to WT (FIG. 3), but this effect was not seen in 3×Tg-AD males. There was also a higher percentage of CD45hi CD11b+ cells in 3×Tg-AD females relative to WT counterparts. There was no difference between 3×Tg-AD and wild-type mice with regard to T cells or B cells in brain.

### Example 3

### Cytokine Production in Peripheral Tissues of 3×Tg-AD Mice

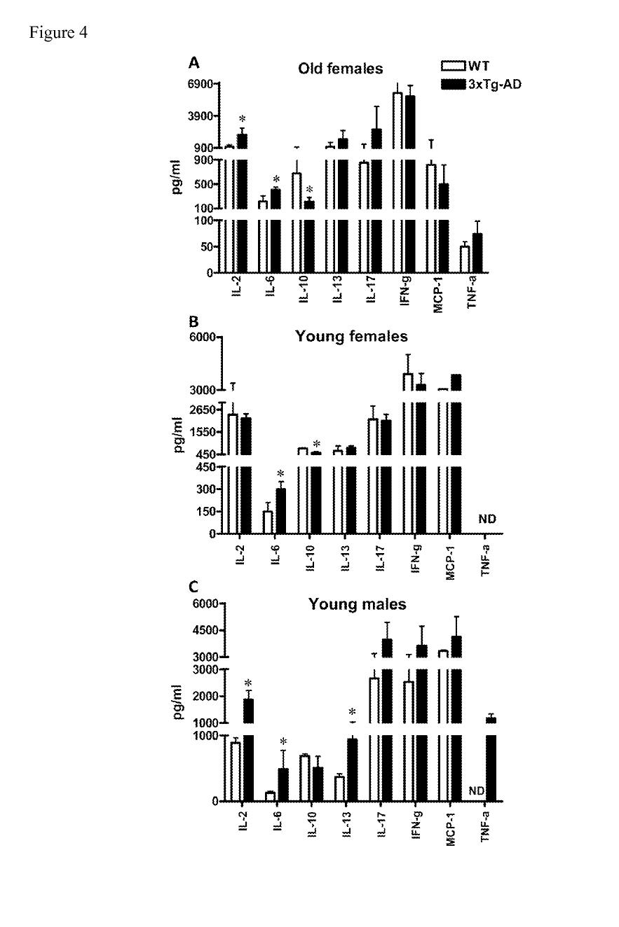

It is of interest to determine if changes in the cellular distribution resulted in an altered cytokine secretion pattern. In FIG. 4, splenocytes from WT and 3×Tg-AD mice were cultured in the presence of plate bound anti-CD3 monoclonal antibody (at 5 μg per plate) and anti-CD28 monoclonal antibody (at 1 μg per plate.) After 24 hours of incubation, supernatants were collected and cytokine expression determined by the Luminex® assay as described in Example 1. Data are presented in the graph as the mean±the standard deviation of 7-9 mice in each group. ND—not detectable. *—statistically significant. Splenocytes from the 12-month-old 3×Tg-AD mice, secreted of IL-2 and IL-6 at a significantly higher rate than age matched WT mice (p<0.01) and secreted IL-10 at ⅓ the rate of the corresponding WT mice—which reached statistical significance. (p<0.01, FIG. 4A). Of note: the 12-month old 3×Tg-AD mice secreted TNF-α at a higher rate than the corresponding WT mice, but this result did not rise to the defined level of significance (p=0.051). Other cytokines tested did not show notable differences between the 12 month and 5-6 month old mice. Splenocytes from 5-6 month old 3×Tg-AD mice secreted significantly more IL-6 than their WT counterparts. This effect was seen in both females (FIG. 4B) and males (FIG. 4C). A similar result was seen in PBMC of 5-6 month old 3×Tg-AD but it rose to the defined level of significance only in female mice.

There was significantly less IL-10 produced in cultures of cells from the 5-6 month old 3×Tg-AD females relative to WT—an effect that was not seen was not seen in 5-6 month old 3×Tg-AD males, and a significantly more IL-13 produced in cultures of cells from 5-6 month old 3×Tg-AD males relative to wild type—an effect that was not seen in 5-6 month old 3×Tg-AD females.

### Example 4

### Gene Expression in 3×Tg-AD Brain and Spleen Tissue

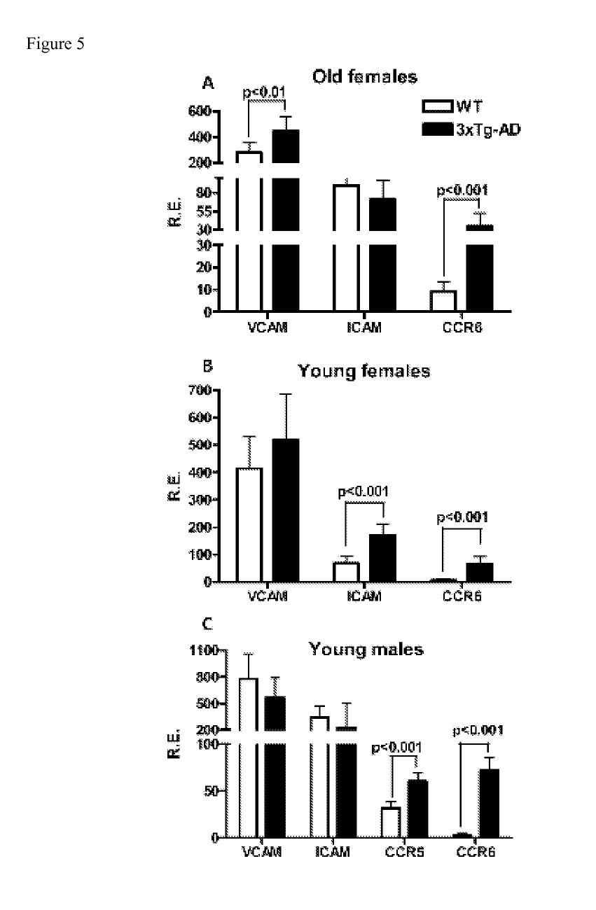

To further establish an inflammatory AD profile, expression of mRNA in brain and spleen tissue for expression of adhesion molecules (ICAM-1, VCAM-1, dysferlin), cytokines (IL-1β, IL-2, IL-6, IL-10, IL-17α, TNF-α), chemokines and receptors (CCL20, CCR2, CCR3, CCR4, CCR5, CCR6, CCR7, CCR8), and the Treg marker, Foxp3 was determined. In FIG. 5, Brains were collected from 12-15 month old (Old) WT and 3×Tg-AD females (data in 5A), 5-6 month old (Young) WT and 3×Tg-AD females (data in 5B), and 5-6 month old WT and 3×Tg-AD males (data in 5C). mRNA was isolated and analyzed by reverse-transcription PCR in triplicate wells. Relative expression (R.E.) of the indicated biomarkers are shown relative to expression of a housekeeping gene (β-actin). Data are presented as the mean±the standard deviation of 3 mice per group for the 12-15 month mice and 3-4 mice per group for the 5-6 month old male and female mice.

As shown in FIG. 5A, brain tissue from older symptomatic 3×Tg-AD female mice exhibited highly elevated and statistically significant expression of CCR6 (p<0.001) and VCAM (p<0.01) compared to brain tissue from age- and gender-matched WT mice. A similar difference in CCR6 expression was also detected in brain tissue from 3×Tg-AD females (p<0.001, FIG. 5B) and males (p<0.001, FIG. 5C), indicating an ongoing inflammatory process in the CNS prior to the onset of AD symptoms. To determine if elevated CCR6 gene expression in the brains of old and young 3×Tg-AD mice relative to WT might also be detected systemically, CCR6 mRNA expression was assessed in spleens from the same mice.

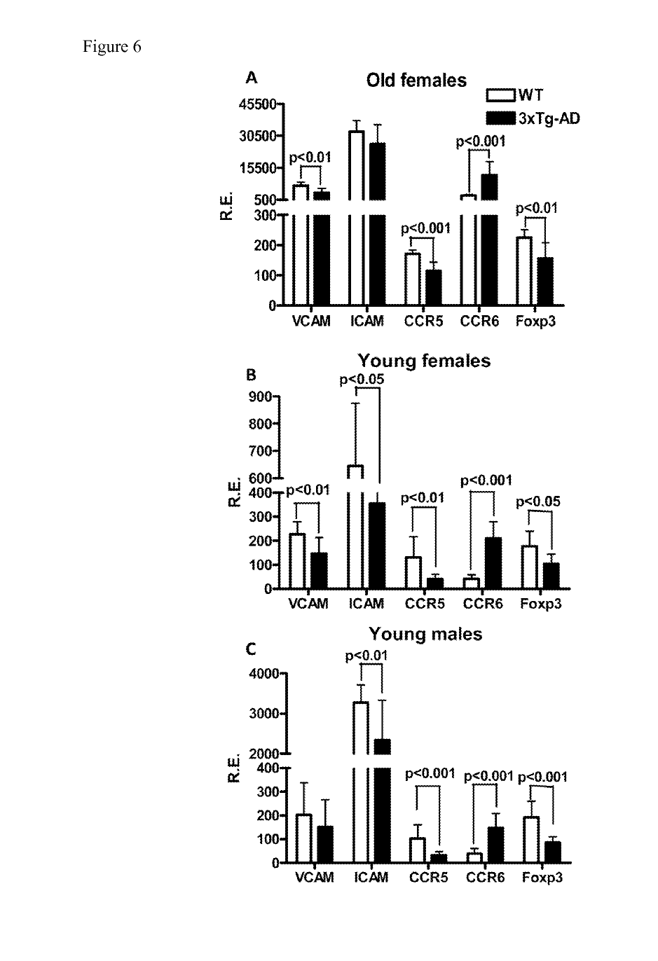

In FIG. 6, spleens were collected from 12-15 month old (Old) WT and 3×Tg-AD females (data in 5A), 5-6 month old (Young) WT and 3×Tg-AD females (data in 5B), and 5-6 month old WT and 3×Tg-AD males (data in 5C). mRNA was isolated and analyzed by reverse transcription PCR in triplicate wells. Relative expression (R.E.) of the indicated biomarkers are shown relative to expression of a housekeeping gene (β-actin). Data are presented as the mean±the standard deviation of 3 mice per group for the 12-15 month mice and 3-4 mice per group for the 5-6 month old male and female mice. Elevated and statistically significant splenic CCR6 expression was observed from the 12-15 month old symptomatic females (p<0.001, FIG. 6A) as well as the 5-6 month old pre-symptomatic females (p<0.001, FIG. 6B) and 5-6 month old pre-symptomatic males (p<0.001, FIG. 6C) compared to WT controls. These data indicate that the elevated CCR6 expression in 3×Tg-AD mice occurs systemically prior to onset of AD-like symptoms, implicating CCR6 as a possible biomarker that signifies that the subject has AD. Other differences in gene expression were detected in spleen but not brain of both older and younger 3×Tg-AD mice relative to age- and gender-matched WT controls. These include including suppressed expression of CCR5 and Foxp3, and significant or directionally suppressed expression of VCAM and ICAM (FIG. 6). There was no difference in splenic mRNA expression of IL-1β, IL-2, IL-6, IL-10, IL-17α, TNF-α, CCR2, CCR3, CCR4, CCR7, CCR8, CCL20 and dysferlin between 3×Tg-AD and WT mice.

### Example 5

### Elevated CCR6 Expression in Human Subjects with Alzheimer's Disease Relative to Healthy Controls

Because CCR6 was expressed systemically and expressed prior to the onset of symptoms in the 3×Tg-AD mouse model, it is a highly promising biomarker of AD. It was therefore selected for testing in human subjects.

After obtaining patient consent, 10 ml blood was collected in heparinized tubes from age matched female healthy controls (HC) and Alzheimer's (AD) subjects. Alzheimer's disease subjects were identified as having AD through the NINDS-ADRDA criteria (See McKahnn et al, Neurology 34, 939-944 (1984) hereby incorporated by reference in its entirety.) Patients were first assessed by an individual clinician, and then a consensus diagnosis was made at a conference of physicians at the Oregon Alzheimer's Disease Center. Peripheral blood mononuclear cells (PBMCs) were separated using Ficoll Paque™ Plus (GE Healthcare) and the buffy coat (containing the leukocyte population) was harvested.

The 10 ml of blood is added to a 50 ml tube, diluted 1:1 in RPMI medium and then 20 ml of Ficoll-Pacque is overlaid upon the diluted blood. The tube is transferred to an appropriate centrifuge and spun at 1600 rpm for 30 minutes (no brake). Cells are harvested from the interface of the Ficoll and liquid layer.

Cells are washed twice with RPMI medium. A sample of the cells was collected and the concentration of cells was determined by counting the cells in a hemocytometer. The cells were then resuspended at a concentration of 10 million cells per ml. Cells were then aliquotted into RNAse free Eppendorf tubes at 5 million cells per tube. The tubes were then spun down and the pellets frozen at −80° C. for storage.

mRNA was isolated from the frozen cells using the Qiagen RNeasy® mini kit. (see U.S. Pat. No. 5,234,809, hereby incorporated by reference in its entirety.) 600 μl of RLT buffer with 1% beta-mercaptoethanol was added to the frozen cell pellet. The pellet was resuspended through micropipetting and then the samples were homogenized for 15-30 seconds with a rotor-stator homogenizer. A volume of 70% ethanol roughly equivalent to the volume of RLT and pellet (600-700 μl) was added to the homogenized samples. The samples are mixed well. A volume of up to 700 μl of the sample is transferred to an RNeasy® column The column is placed into a 2 ml collection tube. The column-collection tube assembly was spun for 1 minute at 10,000 rpm (8000×g). If the sample had a volume greater than 700 μl, then the remaining sample was transferred onto the column after the first spin. Then the remaining sample was spun under the same conditions as the first sample. After each spin, the flow-through was discarded.

Once all of the sample had been spun on the column, a volume of 700 μl of RW1 buffer was added to the column. This was spun for 1 minute at 10,000 rpm. The flow through and collection tube was discarded. The RNeasy column was then placed into a new collection tube. A volume of 500 μl of RPE was then placed on the column. The columns were then spun for 1 minute at 10,000 rpm or 8000×g. The flow through was discarded. A second volume of 500 μl of RPE was then placed on the column. The flow through was discarded. The column was spun again at 14000 rpm and the flow through discarded. The column was then placed into a 1.5 ml RNase-free collection tube. A volume of 40 μl of RNAse free water was then pipette into the center of the column membrane. Care was taken not to touch the membrane. The column was allowed to incubate for 1 minute at room temperature (18-27° C.). The column was then spun for 1 minute at 10,000 rpm to elute the RNA. A 1:50 dilution of the sample RNA was analyzed in a spectrophotometer to determine RNA concentration. RNA from each was then diluted so that each sample had the same concentration of RNA.

RNA was reverse transcribed to cDNA using oligo-dT, random hexamers, and Superscript RT II® (Invitrogen, Grand Island, N.Y., USA) kit. A mix of 12 μl 5×DNAse buffer, 0.4 μl RNAsin, 4 μl of DNAse I, and 3.6 μl water per sample was prepared and 20 μl of the mix was added to each 40 μl RNA sample. The samples were spun in a microcentrifuge, incubated at 37° for 20 minutes, and then at 70° for 10 minute. Samples were then chilled and the contents were collected through a brief spin in the microcentrifuge. The caps of the tubes were removed and replaced with new caps.

A mix of 6 μl of oligo-dT and 6 μl of random hexamer primers per sample was prepared and 12 μl of the mix added to each sample. These were incubated at 70° for 10 minutes, then chilled on ice and spun down to collect the liquid sample.

A mix of 12.5 μl 5× concentrated reverse transcription buffer (250 mM Tris-HCl, pH 8.3 at room temperature; 375 mM KCl; 15 mM MgCl2), 6 μl dNTP's, 12 μl 0.1M DTT, 14.4 μl of RNse-free water and 0.6 μl of RNAsin per sample was prepared. A volume of 45 μl of this mix was added to each sample. Then an additional 2.5 μl of RTII Superscript® was added to each sample. Tube caps were replaced with new caps and the samples were briefly spun in a microcentrifuge. Samples were incubated at 45° C. for 50 minutes, then at 70° C. for 15 minutes. Samples were chilled on ice and briefly spun to collect the contents of the tubes. Samples were then stored at −20° C.

Sample cDNA was prepared for real-time PCR using the TaqMan® 7700 system. Sample cDNA was diluted 1:10 in water. A mix of 10 μl 2× TaqMan® PCR mix and 1 μl of primer/probe mix was prepared per sample. 10 μl of the mix was added to 9 μl diluted cDNA in a 96-well plate configured for use in quantitative real-time PCR and 11 μl master mix was added per sample, for a 20 μl total sample volume. The sample volume is to be input into the TaqMan® operating program.

The primer/probe set used to amplify CCR6 cDNA (SEQ ID NO: 3) in this example was the primer/probe set Hs01890706_s1 (Amplicon length 145) obtained from Applied Biosystems. The primer/probe set used to amplify β-actin cDNA (SEQ ID NO. 21) in this example was the primer/probe set Hs99999903_m1 (Amplicon length 171) obtained from Applied Biosystems.

After completion of the real-time PCR run, the data were analyzed using SDS 7000 software. An expression was set at the most linear section of the amplification curve and the Ct value for each value was determined. A quantitative value in expression units was generated using the formula 1.8^{(βACTIN-CCR6)}(100,000), where βACTIN represents the mean Ct of triplicate wells with primers and probe specific for the beta-actin housekeeping gene and where CCR6 represents the mean Ct of triplicate wells using CCR6 primers and probes. Triplicate wells amplifying β-actin and CCR6 were prepared for each sample. A mean and standard deviation of CCR6 expression level was calculated for each subject using this formula. Results for each subject are indicated in the following table.

TABLE 2

| | | Mean CCR6 | St. Dev CCR6 |
|---|---|---|---|
| Subject # | Type | Expression | Expression |
| 036 | HC | 800.6 | 63.4 |
| 037 | HC | 2656.4 | 1070.7 |
| 038 | HC | 1099.1 | 222.3 |
| | Mean HC | 1572.3 | 1077.7 |
| 031 | AD | 5783.4 | 821.7 |
| 032 | AD | 10724.2 | 934.8 |
| 033 | AD | 8363.2 | 1622.3 |
| 034 | AD | 10656.6 | 3047.5 |
| 035 | AD | 18296.5 | 494.8 |
| | Mean AD | 10767.7 | 4711.0 |

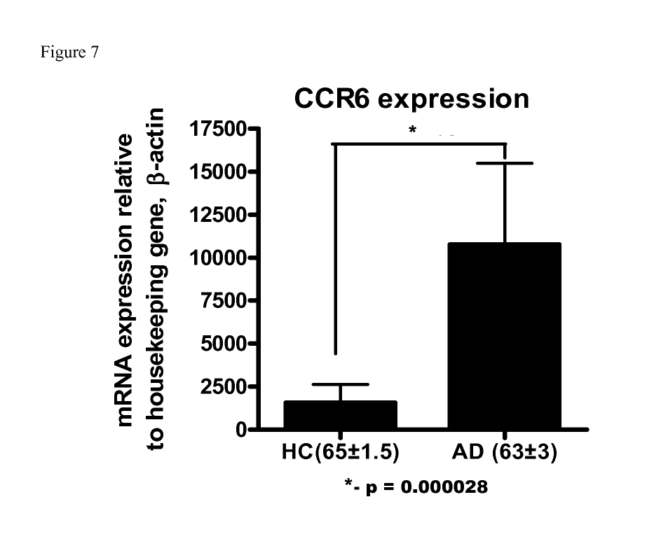

The mean and standard deviation of CCR6 expression for each patient cohort is depicted graphically in FIG. 7. The result is statistically significant. By Student's t-test, the p-value is 0.000028 (the value indicated on FIG. 7). By one-way ANOVA, the p-value is 0.018, and by Mann-Whitney test, the p-value is 0.035. Numbers in parentheses next to the bars are the mean and standard deviation of the ages of the subjects in each cohort.

### Example 6

### Development of a Molecular Diagnostic Test for Alzheimer's Disease Based Upon CCR6 Expression

A test used in diagnosing the presence or absence of Alzheimer's disease in a subject may comprise comparing the expression of CCR6 in a biological sample to a threshold level of expression. Expression of CCR6 in the biological sample that is in excess of the threshold level of expression signifies that the subject from which the biological sample was obtained has AD. Expression of CCR6 below the threshold level of expression signifies that the subject from which the biological sample was obtained does not have, or will not go on to develop AD. Expression of CCR6 at the threshold level or within a calculated range that the test

The nature and numerical value (if any) of the threshold level of expression will vary based on the method chosen to determine the expression of CCR6. The following example illustrates the concept: a threshold level of expression obtained by quantitative reverse transcription PCR will be different than a threshold level of expression obtained by flow cytometry. In the former, the threshold level of expression of CCR6 expression might be derived from Ct relative to the Ct of a housekeeping gene. In the latter, CCR6 expression might be expressed as a percentage of cells staining positively for CCR6. In light of this disclosure, any person of skill in the art would be capable of determining the threshold level of CCR6 expression to determine whether or not a patient has AD using any method of measuring CCR6 expression now known or yet to be disclosed.

The concept of a threshold level of expression should not be limited to a single value or result. Rather, the concept of a threshold level of expression encompasses multiple threshold expression levels that could signify, for example, a high, medium, or low risk that the subject has AD. Alternatively, there could be a low threshold of expression wherein CCR6 expression in the sample below the threshold indicates that the subject does not have AD and a separate high threshold of expression wherein CCR6 expression in the sample above the threshold indicates that the subject does have AD. CCR6 expression in the sample that falls between the two threshold values is inconclusive as to whether the subject has or does not have AD.

To obtain a threshold value of CCR6 expression that indicates that a subject has AD, one would determine CCR6 expression using samples obtained from a first cohort of subjects known to have Alzheimer's disease and from a second cohort known not to have Alzheimer's disease. CCR6 expression is determined in both cohorts and the threshold of CCR6 expression that signifies that a subject is likely to have or will develop AD is determined. Preferably, the threshold level of expression will be the level(s) of expression that provide the maximal ability to predict whether or not a subject has AD on the basis of CCR6 expression and will minimize the number of false positive results and false negative results. The predictive power a threshold level of expression may be evaluated by any of a number of statistical methods known in the art. One of skill in the art will understand which statistical method to select on the basis of the method of determining CCR6 expression and the data obtained. Examples of such statistical methods include:

Receiver Operating Characteristic curves, or “ROC” curves, may be calculated by plotting the value of a variable versus its relative frequency in each of two populations. Using the distribution, a threshold is selected. The area under the ROC curve is a measure of the probability that the expression correctly indicates the diagnosis. If the distribution of CCR6 expression between the two cohorts overlap, then CCR6 expression values from subjects falling into the area of overlap then the subject providing the sample cannot be diagnosed. See, e.g., Hanley et al, Radiology 143, 29-36 (1982) hereby incorporated by reference in its entirety. In that case, a low threshold of expression and a high threshold of expression may be selected.

An odds ratio measures effect size and describes the amount of association or non-independence between two groups. An odds ratio is the ratio of the odds that CCR6 expression above the threshold will occur in samples from a cohort of subjects known to have or who go on to develop AD over the odds that CCR6 expression above the threshold will occur in samples from a cohort of subjects known not to have or who will not go on to develop AD. An odds ratio of 1 indicates that CCR6 expression above the threshold is equally likely in both cohorts. An odds ratio greater or less than 1 indicates that expression of the marker is more likely to occur in one cohort or the other.

A hazard ratio may be calculated by estimate of relative risk. Relative risk is the chance that a particular event will take place. For example: a relative risk may be calculated from the ratio of the probability that samples that exceed a threshold level of expression of CCR6 will be from patients that have AD over the probability that samples that do not exceed the threshold will be from patients that have AD. In the case of a hazard ratio, a value of 1 indicates that the relative risk is equal in both the first and second groups and that the assay has little or no predictive value; a value greater or less than 1 indicates that the risk is greater in one group or another, depending on the inputs into the calculation.

Multiple threshold levels of expression may be selected by so-called “tertile,” “quartile,” or “quintile” analyses. In these methods, multiple groups can be considered together as a single population, and are divided into 3 or more bins having equal numbers of individuals. The boundary between two of these “bins” may be considered threshold levels of expression indicating a particular level of risk that the subject has or will develop AD. A risk may be assigned based on which “bin” a test subject falls into.

The threshold level of expression may also differ based on the purpose of the test. For a test to determine whether or not a subject has or does not have AD, two cohorts of subjects may be tested: one cohort of subjects known to have AD, and another known not to have AD. CCR6 expression is determined by the same method in both cohorts, and the threshold level of expression to differentiate the cohorts is determined.

In another example, a single group of subjects all of whom are asymptomatic for AD is selected. CCR6 expression is determined by the same method in all individuals. After a period of time, the group is divided into two cohorts: one cohort of subjects who developed AD symptoms during the period of time and another cohort of subjects who did not develop AD symptoms. The threshold level of expression to differentiate the two cohorts is determined.

In another example, two cohorts of subjects may be tested: one cohort of subjects known to have AD, and another known not to have AD, but known to have another form of dementia. CCR6 expression is determined by the same method in both cohorts and the threshold level of expression to differentiate the cohorts is determined.

In another example, a single group of subjects, all of whom are asymptomatic for AD are tested for the presence of a genomic polymorphism that indicates that the subject has a predisposition to developing AD. One such genomic allele is ApoE4 (see Strittmatter et al, Proc. Nat. Acad. Sci. USA 90, 8098-8012 (1993), hereby incorporated by reference in its entirety.) Then the group is divided into cohorts on the basis of the presence or absence of ApoE4. CCR6 expression is tested in the ApoE4+ individuals. After a period of time, the ApoE4+ cohort is divided into two subcohorts: one cohort of ApoE4+ subjects who developed AD during the period of time and another cohort of ApoE4+ subjects who did not develop AD during the period of time. The threshold level of expression to differentiate the two subcohorts is determined.

In another example, a single group of subjects, all of whom are asymptomatic for AD are selected. CCR6 expression is determined in all subjects. After a period of time, CCR6 expression is again determined in all subjects. Such a study may be repeated for any number of cycles. A correlation of CCR6 expression over time with development of AD symptoms following such a study could be used to determine a timecourse of CCR6 expression in relation to the development of AD.

### Example 7

### Diagnostic Test for Alzheimer's Disease

This example describes an exemplary diagnostic test, for example in a clinical setting, for detecting Alzheimer's disease in a subject. However, one skilled in the art will appreciate that methods that deviate from these specific methods can also be used to successfully detect Alzheimer's disease in a subject.

In some embodiments, the test includes directly determining an amount of CCR6 in a sample from a subject. The results of the test are provided to a user (such as a clinician or other health care worker, laboratory personnel, or patient) in a perceivable output that provides information about the results of the test. In some examples, the output can be a paper output (for example, a written or printed output), a display on a screen, a graphical output (for example, a graph, chart, voltammetric trace, or other diagram), or an audible output.

In other examples, the output is a numerical value, such as an amount of CCR6 protein in the sample or a relative amount of CCR6 protein in the sample as compared to a control. In additional examples, the output is a graphical representation, for example, a graph that indicates the value (such as amount or relative amount) of CCR6 protein in the sample from the subject on a standard curve. In a particular example, the output (such as a graphical output) shows or provides a cut-off value or level that indicates AD or a predisposition for developing AD if the value or level of the protein in the sample is above the cutoff and absence of AD or a predisposition for developing AD if the value or level of CCR6 protein in the sample is below the cut-off. In some examples, the output is communicated to the user, for example by providing an output via physical, audible, or electronic means (for example by mail, telephone, facsimile transmission, email, or communication to an electronic medical record).

The output can provide quantitative information (for example, an amount of CCR6 protein or an amount of CCR6 protein relative to a control sample or value) or can provide qualitative information (for example, a diagnosis of presence or absence of AD, a likelihood of AD, or a prognosis of AD). In additional examples, the output can provide qualitative information regarding the relative amount of CCR6 protein in the sample, such as identifying presence of an increase in CCR6 protein relative to a control, a decrease in CCR6 protein relative to a control, or no change in CCR6 protein relative to a control.

In some examples, the output is accompanied by guidelines for interpreting the data, for example, numerical or other limits that indicate the presence or absence of AD or a predisposition to developing AD. The guidelines need not specify whether AD is present or absent, although it may include such a diagnosis. The indicia in the output can, for example, include normal or abnormal ranges or a cutoff, which the recipient of the output may then use to interpret the results, for example, to arrive at a diagnosis, prognosis, or treatment plan. In other examples, the output can provide a recommended therapeutic regimen (for example, based on the amount of CCR6 or the amount of increase of CCR6 relative to a control).

### Example 8

### Detection of Reduction of CCR6 Expression in a Mouse Model of Alzheimer's Disease after Administration of a Treatment for Alzheimer's Disease

This example describes efficacy testing of treatments for Alzheimer's disease as measured by a reduction in the amount of CCR6 expressed in a mouse model of Alzheimer's disease. Although particular methods, dosages, and modes of administrations are provided, one skilled in the art will appreciate that variations can be made without substantially affecting the treatment.

Using the 3×Tg-AD mouse model of Alzheimer's disease, cohorts of mice are treated with one or more of the treatments for Alzheimer's disease described in the description of several embodiments or vehicle control. The treatments for Alzheimer's disease can be administered at doses of 1 μg/kg body weight to about 1 mg/kg body weight per dose, such as 1 μg/kg body weight-100 μg/kg body weight per dose, 100 μg/kg body weight-500 μg/kg body weight per dose, or 500 μg/kg body weight-1000 μg/kg body weight per dose. The agent can be administered in several doses, for example continuously, daily, weekly, or monthly. The mode of administration can be any used in the art. The amount of agent administered can be determined by a clinician, and may depend on the particular subject treated. Specific exemplary amounts are provided herein (but the disclosure is not limited to such doses).

Peripheral blood and/or spleen tissue samples are obtained and examined for CCR6 protein and/or mRNA expression. In some examples, the mice are treated one to four times daily with treatments for Alzheimer's disease at a concentration of between 0.02 μg/gram body weight to 1.0 g/gram body weight for between one day and fifty days.

### Example 9

### Detection of Reduction of CCR6 Expression in Patient Samples after a Treatment for Alzheimer's Disease

This example describes efficacy testing of treatments for Alzheimer's disease in samples obtained from patients. Although particular methods, dosages, and modes of administrations are provided, one skilled in the art will appreciate that variations can be made without substantially affecting the treatment.

Subjects are selected that have or are suspected of having Alzheimer's disease based upon their display of symptoms and/or clinical criteria or a predisposition to developing Alzheimer's disease, for example based upon the subject's having a genomic polymorphism that predisposes to AD such as the ApoE4 allele. Briefly, the method can include screening subjects to determine if they have high levels of CCR6 expression using the methods disclosed herein. Subjects having high levels of CCR6 expression are selected.

Following subject selection, a therapeutic effective dose of a treatment for Alzheimer's disease is administered to the subject. The treatment for Alzheimer's disease can be administered at doses of 1 μg/kg body weight to about 1 mg/kg body weight per dose, such as 1 μg/kg body weight-100 μg/kg body weight per dose, 100 μg/kg body weight-500 μg/kg body weight per dose, or 500 μg/kg body weight-1000 μg/kg body weight per dose. However, the particular dose can be determined by a skilled clinician. The agent can be administered in several doses, for example continuously, daily, weekly, or monthly. The mode of administration can be any used in the art. The amount of agent administered to the subject can be determined by a clinician, and may depend on the particular subject treated. Specific exemplary amounts are provided herein (but the disclosure is not limited to such doses).

Biological samples are obtained from subjects, such as subject prior to administration with a treatment for Alzheimer's disease, after administration with a treatment for Alzheimer's disease. The biological sample is examined for CCR6 expression to determine if the subject is responsive to the treatment for Alzheimer's disease.

### Example 10

### Diagnosing a Human Subject with Alzheimer's Disease

This example describes a method of diagnosing a human subject with Alzheimer's disease by detecting CCR6 mRNA and/or protein expression.

A blood sample is taken from a subject who is exhibiting one or more symptoms associated with Alzheimer's disease or from one that is believed to be at risk of developing Alzheimer's disease. Symptoms associated with Alzheimer's disease include the following: (1) short term memory loss (such as forgetting recently learned information); (2) challenges in planning or solving problems; (3) difficulty completing familiar tasks at home, at work or at leisure; (4) confusion with time or place; (5) trouble understanding visual images and spatial relationships; (6) new problems with words in speaking or writing; (7) misplacing things and losing the ability to retrace steps; (8) change in personality or mood; (9) decreased or poor judgment and (10) and withdrawal from work or social activities. CCR6 mRNA and/or protein expression is then measured in the sample. CCR6 mRNA may also be measured in a control sample (such as a blood sample taken from an age- and gender-matched control that is known not to either be at risk of acquiring Alzheimer's disease or display one or more symptoms associated with such disease) by any method known in the art or yet to be disclosed.

While this disclosure has been described with an emphasis upon particular embodiments, it will be obvious to those of ordinary skill in the art that variations of the particular embodiments can be used, and it is intended that the disclosure may be practiced otherwise than as specifically described herein. Features, characteristics, compounds, chemical moieties, or examples described in conjunction with a particular aspect, embodiment, or example of the disclosure are to be understood to be applicable to any other aspect, embodiment, or example of the disclosure. Accordingly, this disclosure includes all modifications encompassed within the spirit and scope of the disclosure as defined by the following claims.

What is claimed is:

1. A method of diagnosing Alzheimer's disease in a subject, the method comprising:
obtaining a biological sample from the subject, wherein the biological sample comprises RNA from mononuclear cells;
contacting the biological sample with an oligonucleotide complementary to part of SEQ ID NO: 3 or a homolog thereof;
subjecting the biological sample to conditions that cause nucleic acid amplification
determining an expression level SEQ ID NO: 3 from the nucleic acid amplification; and
comparing the expression level of SEQ ID NO: 3 in the biological sample to a threshold level of expression,
wherein an expression level in the sample that is equal to or greater than the threshold level of expression signifies that the subject has Alzheimer's disease.

2-17. (canceled)

18. The method of claim 1 wherein the expression level of the biomarker is calculated relative to the expression level of a housekeeping gene in the biological sample.

19. The method of claim 17 wherein the housekeeping gene is SEQ ID NO. 21.

20. The method of claim 19 wherein the threshold level of expression is greater than or equal to 1573 expression units wherein the level of expression is determined by TaqMan® analysis and wherein expression units are calculated by the formula 1.8^{(β-CCR6)}(100,000), wherein β represents the mean Ct of one or more reactions amplifying part of SEQ ID NO. 21 and wherein CCR6 represents the mean Ct of one or more reactions amplifying part of SEQ ID NO: 3.

21-37. (canceled)

38. A kit that facilitates the diagnosis of Alzheimer's disease in a subject, the kit comprising:
a first oligonucleotide complementary to SEQ ID NO. 3;
reagents used in nucleic acid amplification; and
an indication of a threshold level of expression of SEQ ID NO: 3 that signifies that the subject has Alzheimer's disease.

39-45. (canceled)

46. The kit of claim 38 wherein the indication of the threshold level of expression comprises a control configured to provide a result similar to that of the threshold level of expression.

47. The method of claim 1 wherein the threshold level of expression comprises the expression level of a positive control and wherein the expression level of the positive control is determined contemporaneously with the expression level of the sample.

48. The kit of claim 38 wherein the first oligonucleotide is a PCR primer.

49. The kit of claim 48 comprising a primer set selected from the group consisting of PRIMER SET NO: 1, PRIMER SET NO: 2, PRIMER SET NO: 3, PRIMER SET NO: 4, AND PRIMER SET NO: 5.

50. The kit of claim 49 comprising a primer/probe set selected from the group consisting of Hs01890706_s1 (Amplicon length 145), Hs99999079 m1 (Amplicon length 73) Hs01853366_s1 (Amplicon length 142), Hs00171121_m1 (Amplicon length 63), PRIMER/PROBE SET NO: 1, PRIMER/PROBE SET NO: 2, and PRIMER/PROBE SET NO: 3.

51. A method of diagnosing Alzheimer's disease in a subject, the method comprising:
obtaining a biological sample from the subject, wherein the biological sample comprises mononuclear cells;
contacting the biological sample with a reagent with binding specificity for a protein of SEQ ID NO: 4 or a homolog thereof thereby creating a complex between the reagent and the protein;
determining the amount of formation of the complex in the biological sample; and
comparing the amount of formation of the complex in the biological sample to a threshold level of complex formation, wherein an amount of complex formation in the biological sample that is equal to or greater than the threshold level of complex formation signifies that the subject has Alzheimer's disease.

52. A kit that facilitates the performance of the method of claim 51, the kit comprising:
a reagent with binding specificity to SEQ ID NO: 4; and
an indication of the threshold level of complex formation that signifies that the subject has Alzheimer's disease.

53. The kit of claim 52 wherein the reagent comprises an antibody.

54. The kit of claim 53 wherein the antibody is selected from the group consisting of MAB195, R6H1, and 11A9.

55. The kit of claim 53 wherein the antibody comprises a label.

56. The kit of claim 55 wherein the label is a fluorescent label and wherein the kit further comprises reagents used in flow cytometry.
